(19) United States
(12) Patent Application Publication
(10) Pub. No.: US 2026/0294535 A1
(43) Pub. Date: Oct. 1, 2026

(54) DISTANCE MEASUREMENT DURING LASER LITHOTRIPSY PROCEDURES

(71) Applicant: Lumenis Ltd., Yokneam Ilit (IL)

(72) Inventors: Amir Finkel, Zichron Yaakov (IL); Saman Naser, Tira (IL); Asya Mikhaylov, Kiryat Gat (IL); Albert Cogan, Kiryat Motzkin (IL)

(73) Assignee: Lumenis Ltd., Yokneam Ilit (IL)

(21) Appl. No.: 19/636,381

(22) Filed: Apr. 1, 2026

Related U.S. Application Data
(60) Provisional application No. 63/781,893, filed on Apr. 1, 2025.

(51) Int. Cl. A61B 18/22; A61B 18/00; A61B 18/20

(52) U.S. Cl. CPC A61B 18/22; A61B 2018/00642; A61B 2018/2065

(57) ABSTRACT

The present disclosure provides a surgical laser system configured to measure a distance between a distal end of an optical fiber in which laser energy is transmitted and a target where parasitic reflections such as from the distal end and proximal end of the optical fiber are accounted for in measuring the distance.

### PRIORITY

This application claims the benefit of priority under 35 USC § 119 to United States Provisional Patent Application Serial No. 63/781,893, filed Apr. 1, 2025, which is incorporated by reference herein in its entirety and for all purposes.

### TECHNICAL FIELD

The present disclosure generally relates to surgical laser systems used in medical or therapeutic procedures. Particularly, but not exclusively, the present disclosure relates to systems and methods to measure the distance between a distal end of an optical fiber and a target.

### BACKGROUND

Introduction of lasers into the medical field and the development of fiber optic technologies that use lasers has opened numerous applications in treatments, diagnostics, therapies, and the like. Such applications range from invasive and non-invasive treatments to endoscopic surgeries and image diagnostics. For instance, in urinary stone treatment, some stones are required to be fragmented into smaller pieces. A technology known as laser lithotripsy may be used for such fragmenting processes.

In an example lithotripsy procedure, a rigid or flexible ureteroscope is placed through the urinary tract for illumination and imaging. Simultaneously, an optical fiber is inserted through a working channel of the ureteroscope, to a target location (e.g., to the location where the stone is present in the bladder, ureter, or kidney). The laser is then activated to fragment the stone into smaller pieces or to dust it.

These treatments require high amounts of accuracy to ensure that the laser is aimed at the right target (stone, tissue, etc.), to achieve the clinical objectives (e.g., stone fragmentation, stone dusting, tissue coagulation, etc.). Further, the procedure efficiency and/or efficacy can be optimized where the distance between the target and the distal end of the optical fiber (e.g., where the laser radiation is emitted) is known.

Thus, there is a need for improved surgical laser systems configured to measure, or estimate, the distance between the target and the distal end of the optical fiber.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter. The present disclosure applies to surgical laser systems with light detectors arranged to detect optical signals and provides to measure light reflected from the distal end of the optical fiber.

Some embodiments of the disclosure can be implemented as a method for a surgical laser system. The method can comprise generating, via a laser source, an interrogation laser beam, the interrogation laser beam comprising a plurality of wavelength components; measuring, via a reference detector, an intensity of the interrogation laser beam at each of the wavelength components; illuminating, via an optical fiber coupled to the surgical laser system, a target with the interrogation laser beam; measuring, via at least one signal detectors, an intensity of a reflected light beam at each of the plurality of wavelength components, wherein the reflected light beam comprises target reflections and parasitic reflections; deriving for each of the measured intensities, a normalized intensity based on the measured intensity and a dark current intensity associated with the measured intensity; deriving for each of the measured intensities of the reflected light beam, a corrected intensity based on the normalized intensity and one or more parasitic intensities; and estimating a distance between a distal end of the optical fiber and the target based on the corrected intensities.

In further embodiments, the method can comprise applying a smoothing filter to each of the measured intensities prior to deriving the normalized intensities.

In further embodiments of the method, the plurality of wavelength components comprises a first wavelength component, a second wavelength component, and a third wavelength component.

In further embodiments of the method, deriving for each of the measured intensities of the reflected light beam, a corrected intensity comprises deriving a first non-calibration corrected signal for the measured intensity of the reflected light beam at the first wavelength component based on the normalized intensity and a first distal facet reflection value, the first distal facet reflection value associated with the first wavelength component; deriving a second non-calibration corrected signal for the measured intensity of the reflected light beam at the second wavelength component based on the normalized intensity and a second distal facet reflection value, the second distal facet reflection value associated with the second wavelength component; deriving a first calibration corrected signal for the measured intensity of the reflected light beam at the first wavelength component based on the normalized intensity, the first distal facet reflection value, the normalized intensity of the reflected light beam at the third wavelength component, and a third distal facet reflection value, the third distal facet reflection value associated with the third wavelength component; and deriving a second calibration corrected signal for the measured intensity of the reflected light beam at the second wavelength component based on the normalized intensity, the second distal facet reflection value, the normalized intensity of the reflected light beam at the third wavelength component, and the third distal facet reflection value.

In further embodiments of the method, the following equation is used to derive, for each of the first and the second non-calibration corrected signals, corrected signals based on a difference between the internal reflections and the last known fiber distal end reflection normalized signals, respectively.

In further embodiments of the method, estimating the distance between the distal end of the optical fiber and the target based on the corrected intensities comprises deriving a non-calibration corrected distance based on the first and second non-calibration corrected signals; and deriving a calibration corrected distance based on the first and second calibration corrected signals.

In further embodiments of the method, the following equation is used to derive a non-calibration corrected distance and a calibration corrected distance based on fundamental physical equations incorporating the difference between water absorption coefficients of the first wavelength component and the second wavelength component.

In further embodiments, the method can comprise determining that the estimated distance is zero (o); determining that the estimated distance is infinity; selecting the non-calibration corrected distance as the estimated distance; or selecting the calibration corrected distance as the estimated distance.

In further embodiments, the method can comprise determining whether the non-calibration corrected distance is greater than zero; determining whether the calibration corrected distance is greater than zero; and determining that the estimated distance is zero based on a determination that the non-calibration corrected distance is not greater than zero and a determination that the calibration corrected distance is not greater than zero.

In further embodiments, the method can comprise determining whether the non-calibration corrected distance is less than a maximum measurable distance; determining whether the calibration corrected distance is less than a maximum measurable distance; and determining that the estimated distance is infinity based on a determination that the non-calibration corrected distance is not less than the maximum measurable distance and a determination that the calibration corrected distance is not less than the maximum measurable distance; or determining that the estimated distance is infinity based on a determination that the calibration corrected distance is less than zero and a determination that the non-calibration corrected distance is not less than the maximum measurable distance.

In further embodiments, the method can comprise selecting the non-calibration corrected distance as the estimated distance based on a determination that the calibration corrected distance is not greater than zero, a determination that the non-calibration corrected distance is greater than zero, and a determination that the non-calibration corrected distance is less than the maximum measurable distance.

In further embodiments, the method can comprise deriving a calibration ratio based on the normalized intensity of the reflected light beam at the third wavelength component and the third distal facet reflection value; determining whether the calibration ratio is greater than a calibration ratio threshold; and selecting the non-calibration corrected distance as the estimated distance based on a determination that the calibration ratio is greater than the calibration ratio threshold, and a determination that the calibration corrected distance is greater than zero and a determination that the calibration corrected distance is not greater than the maximum measurable distance, or a determination that the calibration corrected distance is greater than the maximum measurable distance and a determination that the non-calibration corrected distance is not greater than the maximum measurable distance; or selecting the calibration corrected distance as the estimated distance based on a determination that the calibration ratio is not greater than the calibration ratio threshold, and a determination that the calibration corrected distance is greater than zero and a determination that the calibration corrected distance is not greater than the maximum measurable distance, or a determination that the calibration corrected distance is greater than the maximum measurable distance and a determination that the non-calibration corrected distance is not greater than the maximum measurable distance.

In further embodiments of the method, the first wavelength component has a lower water absorption coefficient than the second wavelength component.

In further embodiments of the method, the first wavelength component is 1310 nanometers (nm), wherein the second wavelength component is 1340 nm, and wherein the third wavelength component is 1431 nm.

Some embodiments of the disclosure can be implemented as a surgical laser system. The surgical laser system can comprise at least one laser source configured to output an interrogation laser beam, the interrogation laser beam comprising a plurality of wavelength components; a reference detector configured to measure an intensity of the interrogation laser beam at each of the plurality of wavelength components; at least one signal detector configured to measure an intensity of a reflected light beam at each of the plurality of wavelength components; a processor; and a memory comprising instructions, which when executed by the processor cause the surgical laser system to generate, via one or more of the at least one laser source, an interrogation laser beam, the interrogation laser beam comprising a plurality of wavelength components; measure, via the reference detector, an intensity of the interrogation laser beam at each of the wavelength components; illuminate, via an optical fiber coupled to the surgical laser system, a target with the interrogation laser beam; measure, via the at least one signal detector, an intensity of a reflected light beam at each of the plurality of wavelength components, wherein the reflected light beam comprises target reflections and parasitic reflections; derive for each of the measured intensities, a normalized intensity based on the measured intensity and a dark current intensity associated with the measured intensity; derive for each of the measured intensities of the reflected light beam, a corrected intensity based on the normalized intensity and one or more parasitic intensities; and estimate a distance between a distal end of the optical fiber and the target based on the corrected intensities.

In further embodiments of the surgical laser system, the instructions when executed by the processor further cause the surgical laser system to apply a smoothing filter to each of the measured intensities prior to deriving the normalized intensities.

In further embodiments of the surgical laser system, the first wavelength component has a lower water absorption coefficient than the second wavelength component.

In further embodiments of the surgical laser system, the plurality of wavelength components comprises a first wavelength component, a second wavelength component, and a third wavelength component.

In further embodiments of the surgical laser system, the first wavelength component is 1310 nanometers (nm), wherein the second wavelength component is 1340 nm, and wherein the third wavelength component is 1431 nm.

In further embodiments of the surgical laser system, the instructions when executed by the processor further cause the surgical laser system to derive a first non-calibration corrected signal for the measured intensity of the reflected light beam at the first wavelength component based on the normalized intensity and a first distal facet reflection value, the first distal facet reflection value associated with the first wavelength component; derive a second non-calibration corrected signal for the measured intensity of the reflected light beam at the second wavelength component based on the normalized intensity and a second distal facet reflection value, the second distal facet reflection value associated with the second wavelength component; derive a first calibration corrected signal for the measured intensity of the reflected light beam at the first wavelength component based on the normalized intensity, the first distal facet reflection value, the normalized intensity of the reflected light beam at the third wavelength component, and a third distal facet reflection value, the third distal facet reflection value associated with the third wavelength component; and derive a second calibration corrected signal for the measured intensity of the reflected light beam at the second wavelength component based on the normalized intensity, the second distal facet reflection value, the normalized intensity of the reflected light beam at the third wavelength component, and the third distal facet reflection value.

In further embodiments of the surgical laser system, the instructions when executed by the processor further cause the surgical laser system to derive, for each of the first and the second non-calibration corrected signals, corrected signals based on a difference between the internal reflections and the last known fiber distal end reflection normalized signals, respectively.

In further embodiments of the surgical laser system, the instructions when executed by the processor further cause the surgical laser system to derive a non-calibration corrected distance and a calibration corrected distance based on fundamental physical equations incorporating the difference between water absorption coefficients of the first wavelength component and the second wavelength component.

In further embodiments of the surgical laser system, the instructions when executed by the processor further cause the surgical laser system to determine that the estimated distance is zero (o); determine that the estimated distance is infinity; select the non-calibration corrected distance as the estimated distance; or select the calibration corrected distance as the estimated distance.

In further embodiments of the surgical laser system, the instructions when executed by the processor further cause the surgical laser system to determine whether the non-calibration corrected distance is greater than zero; determine whether the calibration corrected distance is greater than zero; and determine that the estimated distance is zero based on a determination that the non-calibration corrected distance is not greater than zero and a determination that the calibration corrected distance is not greater than zero.

In further embodiments of the surgical laser system, the instructions when executed by the processor further cause the surgical laser system to determine whether the non-calibration corrected distance is less than a maximum measurable distance; determine whether the calibration corrected distance is less than a maximum measurable distance; and determine that the estimated distance is infinity based on a determination that the non-calibration corrected distance is not less than the maximum measurable distance and a determination that the calibration corrected distance is not less than the maximum measurable distance; or determine that the estimated distance is infinity based on a determination that the calibration corrected distance is less than zero and a determination that the non-calibration corrected distance is not less than the maximum measurable distance.

In further embodiments of the surgical laser system, the instructions when executed by the processor further cause the surgical laser system to select the non-calibration corrected distance as the estimated distance based on a determination that the calibration corrected distance is not greater than zero, a determination that the non-calibration corrected distance is greater than zero, and a determination that the non-calibration corrected distance is less than the maximum measurable distance.

In further embodiments of the surgical laser system, the instructions when executed by the processor further cause the surgical laser system to derive a calibration ratio based on the normalized intensity of the reflected light beam at the third wavelength component and the third distal facet reflection value; determine whether the calibration ratio is greater than a calibration ratio threshold; and selecting the non-calibration corrected distance as the estimated distance based on a determination that the calibration ratio is greater than the calibration ratio threshold, and a determination that the calibration corrected distance is greater than zero and a determination that the calibration corrected distance is not greater than the maximum measurable distance, or a determination that the calibration corrected distance is greater than the maximum measurable distance and a determination that the non-calibration corrected distance is not greater than the maximum measurable distance; or select the calibration corrected distance as the estimated distance based on a determination that the calibration ratio is not greater than the calibration ratio threshold, and a determination that the calibration corrected distance is greater than zero and a determination that the calibration corrected distance is not greater than the maximum measurable distance, or a determination that the calibration corrected distance is greater than the maximum measurable distance and a determination that the non-calibration corrected distance is not greater than the maximum measurable distance.

Some embodiments of the disclosure can be implemented as at least one non-transitory machine-readable storage device comprising instructions, which when executed by a processor of a surgical laser system cause the surgical laser system to generate, via one or more of laser sources of the surgical laser system, an interrogation laser beam, the interrogation laser beam comprising a plurality of wavelength components; measure, via a reference detector of the surgical laser system, an intensity of the interrogation laser beam at each of the wavelength components; illuminate, via an optical fiber coupled to the surgical laser system, a target with the interrogation laser beam; measure, via at least one signal detector of the surgical laser system, an intensity of a reflected light beam at each of the plurality of wavelength components, wherein the reflected light beam comprises target reflections and parasitic reflections; derive for each of the measured intensities, a normalized intensity based on the measured intensity and a dark current intensity associated with the measured intensity; derive for each of the measured intensities of the reflected light beam, a corrected intensity based on the normalized intensity and one or more parasitic intensities; and estimate a distance between a distal end of the optical fiber and the target based on the corrected intensities.

In further embodiments of the at least one non-transitory machine-readable storage device, the plurality of wavelength components comprises a first wavelength component, a second wavelength component, and a third wavelength component and wherein the instructions when executed by the processor further cause the surgical laser system to derive a first non-calibration corrected signal for the measured intensity of the reflected light beam at the first wavelength component based on the normalized intensity and a first distal facet reflection value, the first distal facet reflection value associated with the first wavelength component; derive a second non-calibration corrected signal for the measured intensity of the reflected light beam at the second wavelength component based on the normalized intensity and a second distal facet reflection value, the second distal facet reflection value associated with the second wavelength component; derive a first calibration corrected signal for the measured intensity of the reflected light beam at the first wavelength component based on the normalized intensity, the first distal facet reflection value, the normalized intensity of the reflected light beam at the third wavelength component, and a third distal facet reflection value, the third distal facet reflection value associated with the third wavelength component; and derive a second calibration corrected signal for the measured intensity of the reflected light beam at the second wavelength component based on the normalized intensity, the second distal facet reflection value, the normalized intensity of the reflected light beam at the third wavelength component, and the third distal facet reflection value.

In further embodiments of the at least one non-transitory machine-readable storage device, the instructions when executed by the processor further cause the surgical laser system to derive, for each of the first and the second non-calibration corrected signals, corrected signals based on a difference between the internal reflections and the last known fiber distal end reflection normalized signals, respectively.

In further embodiments of the at least one non-transitory machine-readable storage device, the instructions when executed by the processor further cause the surgical laser system to derive a non-calibration corrected distance and a calibration corrected distance based on fundamental physical equations incorporating the difference between water absorption coefficients of the first wavelength component and the second wavelength component.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A illustrates a surgical laser system in accordance with embodiments of the disclosure.

FIG. 1B illustrates a portion of the surgical laser system of FIG. 1A in greater detail.

FIG. 2 illustrates an interrogation sub-system in accordance with embodiments of the disclosure.

FIG. 3 illustrates a method for measuring a distance in accordance with embodiments of the disclosure.

FIG. 4 illustrates a table for use with the distance measurement method of FIG. 3.

FIG. 5 illustrates a method for stabilizing detected signals in accordance with embodiments of the disclosure.

FIG. 6 illustrates a dataflow diagram in accordance with embodiments of the disclosure.

FIG. 7 illustrates a method for accounting for parasitic reflections in accordance with embodiments of the disclosure.

FIG. 8 illustrates another dataflow diagram in accordance with embodiments of the disclosure.

FIG. 9 illustrates another interrogation sub-system in accordance with embodiments of the disclosure.

FIG. 10 illustrates a first method for controlling a surgical laser system based on interpolated damage to an optical fiber in accordance with embodiments of the disclosure.

FIG. 11 illustrates a second method for controlling a surgical laser system based on interpolated damage to an optical fiber in accordance with embodiments of the disclosure.

FIG. 12 illustrates a third method for controlling a surgical laser system based on interpolated damage to an optical fiber in accordance with embodiments of the disclosure.

FIG. 13 illustrates a fourth method for controlling a surgical laser system based on interpolated damage to an optical fiber in accordance with embodiments of the disclosure.

FIG. 14 illustrates a method for injecting interrogation pulses between pulses of a therapeutic laser beam in accordance with embodiments of the disclosure.

FIG. 15 illustrates another method for injecting interrogation pulses between pulses of a therapeutic laser beam in accordance with embodiments of the disclosure.

FIGS. 16, 17, 18, and 19 illustrate different example combined laser beams comprising therapeutic laser beam pulses and interrogation laser beam pulses in accordance with embodiments of the disclosure.

FIG. 20 illustrates a computer-readable storage medium in accordance with embodiment of the disclosure.

FIG. 21 illustrates a specially programmed computing system in accordance with embodiments of the disclosure.

### DETAILED DESCRIPTION

The present disclosure applies to surgical laser systems with light detectors arranged to detect optical signals. An illustrative surgical laser system can be provided with both therapeutic and interrogative laser sources and can be configured to measure the distance between a target and a distal end of an optical fiber using the interrogative laser sources. It is to be appreciated that the primary signal for measuring this distance is light that is reflected from the target. However, this signal is mixed with other parasitic reflections (e.g., reflections from internal optical components, reflections from the proximal end of the optical fiber, and reflections from the distal end of the optical fiber). The present disclosure provides systems and methods to measure and account for these parasitic signals, as well as to account for other phenomena that affect the measurement process.

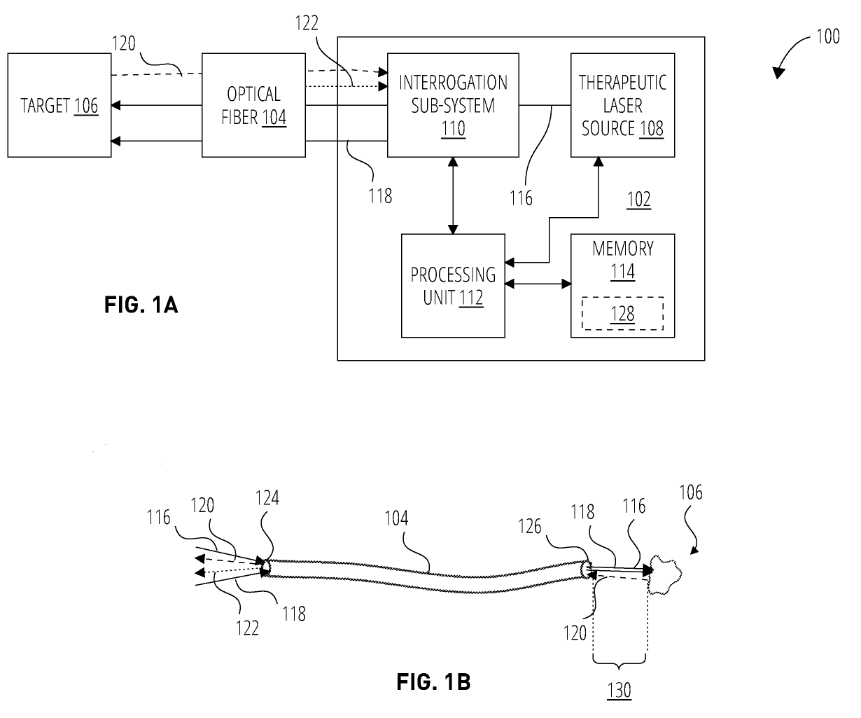

FIG. 1A show an exemplary surgical laser system 100 for estimating a distance between a distal end of an optical fiber and a target, in accordance with some embodiments of the present disclosure. The surgical laser system 100 comprises a surgical laser console 102 and an optical fiber 104. To aid the discussion, the surgical laser system 100 is depicted treating a target 106. In an exemplary lithotripsy procedure, the target 106 can be a stone. However, it will be appreciated that the surgical laser console 102 and optical fiber 104 could be used to treat other types of targets (e.g., tissue, a tumor, a cyst, or the like). With illustrative examples, the target can be disposed in a liquid environment within a subject (e.g., a human, an animal, or the like).

The surgical laser console 102 includes a therapeutic laser source 108, an interrogation sub-system 110, a processing unit 112, and a memory 114. During operation, the therapeutic laser source 108 is configured to generate therapeutic laser pulses 116 while interrogation sub-system 110 is configured to generate interrogation laser pulses 118.

In general, the therapeutic laser source 108 can be any of a variety of laser light sources (e.g., solid-state lasers, gas lasers, diode lasers, and fiber lasers). As a specific example, the therapeutic laser source 108 can be a Holmium (Ho) laser, such as a Ho:YAG laser. In yet another example, the therapeutic laser source 108 can be a Thulium (Tm) fiber laser.

An example of the interrogation sub-system 110 is provided in greater detail below. However, in general the interrogation sub-system 110 is configured to generate interrogation laser pulses 118, which comprise multiple (e.g., two, three, four, etc.) laser beams each having different wavelengths.

The therapeutic laser pulses 116 and the interrogation laser pulses 118 are transmitted towards the target 106 via the optical fiber 104. Target reflected laser pulses 120 are light reflected from the target responsive to the therapeutic laser pulses 116 and/or the interrogation laser pulses 118 being incident on the target as well as light reflected from other features, referred to herein as parasitic reflections 122. This is depicted in greater detail in FIG. 1B, which depicts the optical fiber 104. The optical fiber 104 includes both a proximal end 124 and a distal end 126. The proximal end 124 is the end of the optical fiber 104 coupled to the surgical laser console 102 and through which therapeutic laser pulses 116 and interrogation laser pulses 118 enter while the distal end 126 is the end of the optical fiber 104 through which the therapeutic laser pulses 116 and interrogation laser pulses 118 are emitted. During a procedure, the physician can manipulate the distal end 126 of the optical fiber 104 to “point” the optical fiber 104 at the target 106 and to place the distal end 126 within a desired distance to the target 106.

Further, at outlined above, target reflected laser pulses 120 is light reflected off the target in response to illumination by therapeutic laser pulses 116 and/or interrogation laser pulses 118. The target reflected laser pulses 120 can be received at the distal end 126 of the optical fiber 104 and transmitted to the surgical laser console 102. Other light reflections contaminate the target reflected laser pulses 120. For example, reflections from the distal end 126 of the optical fiber 104, the proximal end 124 of the optical fiber 104, and other internal optical components of the interrogation sub-system 110 (e.g., coupling lenses, etc.). These reflections are collectively referred to herein at parasitic reflections 122.

An example interrogation sub-system 110 is described below. However, the general principle relies on the power of the interrogation laser pulses 118 relative to the power of the target reflected laser pulses 120. To that end, interrogation sub-system 110 includes detectors or other sensors (described below) configured to measure the power of interrogation laser pulses 118 and target reflected laser pulses 120. As such, it is important to account for the parasitic reflections 122 and other phenomena that might affect the measurement.

The processing unit 112 can include circuitry arranged to execute instructions 128 stored in memory 114, which instructions when executed cause the processing unit 112 to send control signals to the interrogation sub-system 110 to measure parasitic reflections 122 (or portions of the parasitic reflections 122) and account for the measured parasitic reflections 122 as part of measuring the distance 130 between the target 106 and the distal end 126 of the optical fiber 104. With some examples, the processing unit 112 and memory 114 may be provided by a computing device (e.g., a laptop, a desktop, a mobile phone, a tablet, or the like).

It is to be appreciated that the surgical laser console 102 and particularly the therapeutic laser source 108 and interrogation sub-system 110 can include a variety of optical components besides the laser sources described above. For example, therapeutic laser source 108 and interrogation sub-system 110 can include (e.g., in different combinations as may be the case) light sources, polarizers, beam splitters, beam combiners, light detector, wavelength division multiplexers, collimators, circulators, etc.

Further, although not depicted, the surgical laser console 102 could include a display and/or other input and/or output devices configured to provide interaction with the surgical laser console 102 by a user (e.g., physician, or the like).

It is to be appreciated that the target 106 is often in a liquid environment (e.g., urine, water, blood, etc.) For example, during a lithotripsy procedure, the target 106 may be a stone and may be disposed in an environment that includes a mixture of urine, irrigation fluid, and blood. Accordingly, the distal end 126 of the optical fiber 104 will also be disposed in the liquid environment during the procedure. When the therapeutic laser pulses 116 are emitted from the distal end 126 of the optical fiber 104, some of the liquid may be vaporized and form a bubble or bubbles.

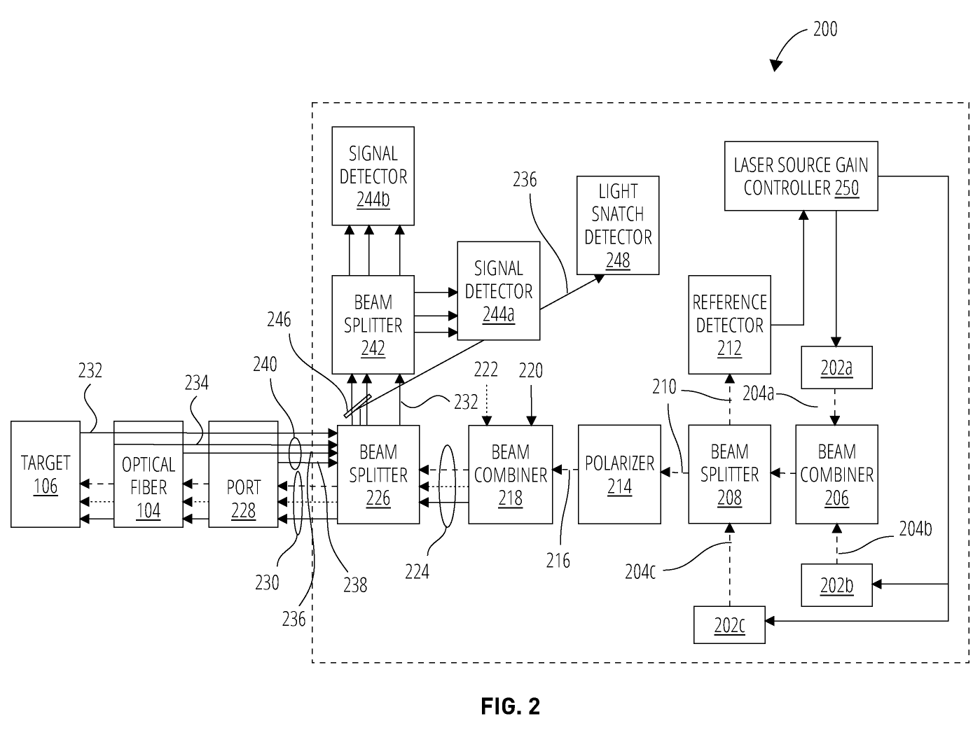

FIG. 2 shows an example interrogation sub-system 200, which can be provided in accordance with some embodiments of the present disclosure. For example, the interrogation sub-system 200 can be provided as the interrogation sub-system 110 of the surgical laser console 102 shown as part of the surgical laser system 100 in FIGS. 1A and 1B.

As depicted, the interrogation sub-system 200 comprises laser sources 202a, 202b, and 202c, which are configured to generate, respectively, laser beams 204a, 204b, and 204c. The laser sources 202a, 202b, and 202care each configured to generate laser beams of a different wavelengths. For example, laser source 202a can be configured to generate laser beam 204a having a first wavelength while the laser source 202b can be configured to generate the laser beam 204b having a second wavelength different than the first wavelength. Of note, the first wavelength can have an absorption coefficient (e.g., in water, or the like) higher than an absorption coefficient of the second wavelength. Further, the laser source 202c can be configured to generate the laser beam 204c having yet another wavelength, different from the wavelength of the laser beams 204a and 204b.

As used herein, the terms “high” and “low” are intended to be interpreted relative to each other, or in the alternative relative to a threshold characteristic describing the water absorption of a particular wavelength. For example, a high water absorption coefficient can be greater than or equal to 150% of the low water absorption coefficient. In a non-limiting example, the laser beam 204a can have a wavelength of approximately 1310 nanometers (nm); laser beam 204b can have a wavelength of approximately 1340 nm. Further, the laser sources may include a calibration laser beam (e.g., approximately 1431 nm, or the like). This is described in greater detail below.

The laser beams 204a, 204b, and 204c are combined via beam combiner 206 and beam splitter 208 into interrogation laser beams 210 (e.g., interrogation laser pulses 118 of FIG. 1A, or the like). A reference detector 212 is associated with and in optical communication with the beam splitter 208. The reference detector 212 is arranged to measure the optical power of each respective component of interrogation laser beams 210 (e.g., component of laser sources 202a, 202b, and 202c).

The interrogation sub-system 200 includes laser source gain controller 250 configured to control the current gain of the laser sources 202a, 202b, and 202c to maintain interrogation laser beams 208 incident on the reference detector 210 at target levels. Operation of interrogation sub-system 200 is described in greater detail below. However, in general, laser source gain controller 250 applies a proportional integral (PI) or PI derivative (PID) algorithm for each laser source based on relative readings on the detector to be stabilized. As such, the laser source gain controller 250 is coupled in a closed loop with the reference detector 210 and the laser sources 202a, 202b, and 202c. As such, in this example, the laser source gain controller 250 is configured to stabilize the signals incident on the reference detector 210 based on adjusting the current gain of the laser sources 202a, 202b, and 202c. Other embodiments where the laser source gain controller 250 is coupled to the signal detectors 244a and/or 244b and configured to stabilize the signals incident on either or both of these detectors or where the laser source gain controller 250 is coupled to the reference detector 212 and the signal detectors 244a and/or 244b and configured to stabilize the signals incident on ones of these detectors are contemplated.

The beam splitter 208 is further associated with and in optical communication with the polarizer 214. The beam splitter 208 is configured to provide a portion of the interrogation laser beams 210 to the polarizer 214. In some embodiments, the polarity of the polarizer 214 may be pre-configured and arranged to output polarized light beam 216. The interrogation sub-system 200 further includes a beam combiner 218, which is in optical communication with the polarizer 214. In such a manner, the polarized light beam 216 obtained as an output from the polarizer 214 is provided as input to the beam combiner 218. The beam combiner 218 may combine the polarized light beams 216 (e.g., corresponding to the interrogation laser beams 210) with a therapeutic laser beam 220 (e.g., therapeutic laser pulses 116 of FIG. 1A, or the like) and an aiming beam 222, to form combined beam 224.

The interrogation sub-system 200 further includes a beam splitter 226 and a port 228. The beam splitter 226 is arranged in optical communication with the beam combiner 218. As such, the beam splitter 226 can receive the combined beam 224 (comprising the polarized light beam 216, the therapeutic laser beam 220, and the aiming beam 222) from the beam combiner 218 and output the combined beam 224 to the port 228. In such a manner, the beam splitter 226 is optically coupled to the optical fiber 104 via the port 228 such that a portion of the combined beam 224 (e.g., denoted as combined beam portion 230) is transmitted through the optical fiber 104 to be incident on the target 106.

When the combined beam portion 230 is incident on the target 106, the target 106 may reflect some portion of the incident combined beam portion 230 away from the optical fiber 104 and some portion of the light towards the optical fiber 104, wherein the portion of light reflected towards the optical fiber 104 may re-enter the optical fiber 104 (e.g., at the distal end 126 of the optical fiber 104). The portion of the reflected light re-entering the optical fiber 104 at the distal end 126 may be referred to as target reflection beam 232. As noted, the target reflection beam 232 may be contaminated and/or mixed with other reflections. For example, some of the combined beam portion 230 may be reflected from the distal facet (e.g., distal end 126) of the optical fiber 104 as distal facet reflection beam 234 while some of the combined beam portions 230 may be reflected from the proximal facet (e.g., proximal end 124) of the optical fiber 104 as proximal facet reflection beam 236. Further, some of the combined beam portion 230 may be reflected from other internal optical components (e.g., port 228, coupling lenses, etc.) as internal reflection beam 238. The collection of distal facet reflection beam 234, proximal facet reflection beam 236, and internal reflection beam 238 are referred to as parasitic reflected beams 240 (e.g., parasitic reflections 122 of FIG. 1A, or the like).

The present disclosure provides to account for the parasitic reflected beams 240 by either eliminating them or measuring them such that the target reflection beam 232 can be isolated during measurement of the distance 130.

The parasitic reflected beams 240 may be subjected to the beam splitter 226, which directs the parasitic reflected beams 240 to another beam splitter 242. The beam splitter 242 may be in optical communication with signal detectors 244a and 244b. In some examples, the beam splitter 242 may be a polarizing beam splitter. As such portions of parasitic reflected beams 240 having one polarity (e.g., P) may be transmitted to one of the signal detectors 244a or 244b while portions of the parasitic reflected beams 240 having the other polarity (e.g., S) may be transmitted to the other signal detector 244a or 244b.

The interrogation sub-system 200 further includes a light snatch mirror 246 disposed between beam splitter 226 and the beam splitter 242 and configured to direct the proximal facet reflection beam 236 to a light snatch detector 248, thereby eliminating the proximal facet reflection beam 236 from reaching the signal detectors 244a and 244a and as a result interfering with the measurement. The complete details of eliminating the proximal facet reflection beam 236 from the parasitic reflected beams 240 is beyond the scope of this disclosure. However, the general principle relies on the fact that the proximal facet reflection beam 236 have a different angular rotation than other parasitic beams, and as such, can be selectively directed towards light snatch detector 248 by light snatch mirror 246 without interfering with the other components of parasitic reflected beams 240.

Thus, only the remaining components of parasitic reflected beams 240 (e.g., distal facet reflection beam 234 and internal reflection beam 238) as well as the target reflection beam 232 reach the signal detectors 244a and 244b. The signal detectors 244a and 244b are configured to measure intensities of the components of parasitic reflected beams 240 and the target reflection beam 232 and to transmit the intensities to the processing unit 112. Accordingly, the processing unit 112 may estimate the distance 130 between the distal end 126 of the optical fiber 104 and the target 106 based on the measured intensities.

Accordingly, interrogation sub-system 200 provides four (4) optical detectors (e.g., reference detector 212, signal detector 244a, signal detector 244b, and light snatch detector 248). Each of these four (4) detectors can be configured to output a signal indicative of an intensity of a component of the overall laser beams. For example, interrogation sub-system 200 is configured to generate interrogation laser beams 210 comprising laser beams 204a, 204b, and 204c. As such, the four (4) detectors are configured to output a signal for each of the respective laser beams 204a, 204b, and 204c. Further, each of the four (4) detectors are configured to output signals indicative of an intensity of a “dark current” (e.g., leakage current measured when all laser sources 202a, 202b, and 202c) are off. With some embodiments, the four (4) detectors can be 24-bit optical detectors to allow sufficient dynamic range to support a wide range of targets 106 having different reflection characteristics and to support fiber distal end degradation during the treatment procedure. As such, there are sixteen (16) signals contemplated herein (e.g., four (4) from each of the four (4) detectors).

It is to be appreciated that for accurate measurements (1) the interrogation laser beams 210 and the associated parasitic reflected beams 240 should travel through the liquid environment (e.g., water, or the like) to take advantage of the difference in absorption coefficients between the components of the interrogation laser beams 210; and (2) the parasitic reflected beams 240 associated with the interrogation laser beams 210 as well as the contribution from the dark current need to either be eliminated or considered.

It is to be appreciated that internal reflection beam 238 are fixed and do not change with time. As such, these signals can be measured once and the measurements used in an overall distance measurement algorithm. Further it is to be appreciated that the distal facet reflection beam 234 changes during the procedure. For example, the surface of the distal end 126 of the optical fiber 104 is continuously damaged due to the energy impact from the therapeutic laser beam 220 and in instances where fragments of the target 106 impact the surface. Changes to the surface of the distal end 126 of the optical fiber 104 have different, or varying, impacts to each of the different components of the interrogation laser beams 210. Additionally, other phenomenon can contribute to the dynamic nature of the distal facet reflection beam 234, such as, for example, bubbles near the surface of the distal end 126 of the optical fiber 104 forming and collapsing will affect the distal facet reflection beam 234.

Since the distal facet reflection beam 234 changes during the procedure, it cannot be measured upfront like the internal reflection beam 238. Further, the amplitude of the changes in the distal facet reflection beam 234 are often higher than that of the target reflection beam 232. As such, the present disclosure provides to measure the distal facet reflection beam 234 and calibrate and/or adjust the distance measurement algorithm based on the measurement of the distal facet reflection beam 234.

With some embodiments, the processing unit 112 can be configured to execute instructions 128 to measure the distance 130 between the distal end 126 of the optical fiber 104 and the target 106 based on signals received from the reference detector 212, the signal detectors 244a and 244b, and the light snatch detector 248. Examples of measuring the distance 130 are provided in greater detail below.

The disclosure provides to periodically measure the distance 130 and further provides to measure the distal facet reflection beam 234 during periods where the distance 130 between the distal end 126 of the optical fiber 104 and the target 106 is great enough such that all of the interrogation laser beams 210 is absorbed by the liquid medium, and therefore, target reflection beam 232 is substantially zero.

As outlined above, proximal facet reflection beam 236 is eliminated and internal reflection beam 238 is known and can thus be subtracted from the parasitic reflected beams 240. Accordingly in periods where the distance 130 between the distal end 126 of the optical fiber 104 and the target 106 are great enough that interrogation laser beams 210 is substantially absorbed by the liquid medium, the entire signal reaching the signal detectors 244a and 244b may be due to the distal facet reflection beam 234.

In a typical lithotripsy procedure, there are many time periods where the target 106 is far enough away from the distal end 126 of the optical fiber 104 (e.g., greater than or equal to 4 millimeters, or the like) such that signals reaching the signal detectors 244a and 244b are originating only from the distal end 126 of the optical fiber 104 (e.g., are only distal facet reflection beam 234).

The processing unit 112 can execute instructions 128 to identify periods where the distance 130 is above a threshold value (e.g., 4 mm, or the like) for a long enough time and can measure the distal facet reflection beam 234 at this time and update and/or calibrate the overall distance 130 measurement algorithm based on this measurement. Accordingly, the distal facet reflection beam 234 can be periodically updated and accounted for during the procedure, which provides a more accurate measurement of the distance 130 between the distal end 126 of the optical fiber 104 and the target 106.

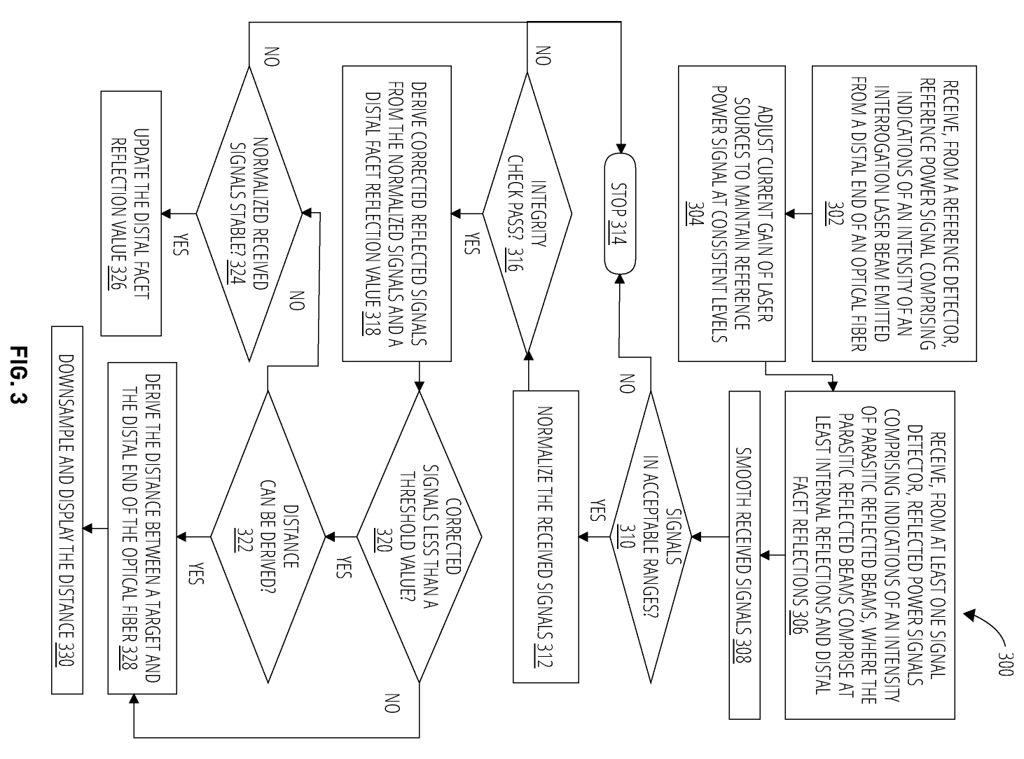

FIG. 3 illustrates a flowchart showing a method 300 for measuring a distance between a distal end of an optical fiber a target. The method 300 can be implemented during a surgical laser procedure, such as a laser lithotripsy procedure. The method 300 is described with reference to the surgical laser system 100 of FIGS. 1A and 1B and to the interrogation sub-system 200 of FIG. 2. It is to be appreciated however, that the method 300 could be implemented by a surgical system different than the one depicted herein.

The method 300 can begin at block 302. At block 302 “receive, from a reference detector, reference power signal comprising indications of an intensity of an interrogation laser beam emitted from a distal end of an optical fiber” a reference power signal comprising indications of an intensity of an interrogation laser beam emitted from a distal end of an optical fiber can be received. For example, processing unit 112 can execute instructions 128 to receive from reference detector 212, signals comprising indications of the power of interrogation laser beams 210. As noted above, interrogation laser beams 210 can comprise multiple components (e.g., laser beams 204a, 204b, 204c, etc.) In some examples, processing unit 112 can execute instructions 128 to receive indications of a power of each respective component.

Continuing to block 304 “adjust current gain of laser sources to maintain reference power signal at consistent levels” the current gain of laser sources can be adjusted to maintain reference power signals at consistent levels. For example, processing unit 112 can execute instructions 128 to adjust the current gain of the laser sources 202a, 202b, and 202c so that the signals output from the reference detector 212 corresponding to these laser sources are maintained at consistent levels.

Continuing to block 306 “receive, from at least one signal detector, reflected power signals comprising indications of an intensity of parasitic reflected beams, where the parasitic reflected beams comprise at least internal reflections and distal facet reflections” reflected power signals comprising indications of an intensity of parasitic reflected beams can be received, where the parasitic reflected beams comprise at least internal reflections and distal facet reflections. For example, processing unit 112 can execute instructions 128 to receive from signal detectors 244a and/or 244b, signals comprising indications of the power of parasitic reflected beams 240. As noted above, parasitic reflected beams 240 can comprise multiple components (e.g., distal facet reflection beam 234, proximal facet reflection beam 236, internal reflection beam 238, etc.) In some examples, proximal facet reflection beam 236 can be optically eliminated from the parasitic reflected beams 240 (e.g., by light snatch mirror 246 and light snatch detector 248, or the like). As such, signals generated by signal detectors 244a and 244b will depend only on distal facet reflection beam 234 and internal reflection beam 238. Further, as introduced above there will be periods of time during a procedure where the target 106 is far enough away from the distal end 126 of the optical fiber 104 that all of interrogation laser beams 210 will be absorbed by the liquid medium, and as such, target reflection beam 232 will be substantially zero, or will be small enough to allow calibration as described herein.

Continuing to block 308 “smooth received signals” the received signals can be smoothed. For example, processing unit 112 can execute instructions 128 to apply a filter to reduce noise. With some embodiments, processing unit 112 can execute instructions 128 to apply a first order low pass filter (LPF) to the received signals. However, it is to be appreciated that other types of smoothing algorithms may be applied to filter and/or reduce noise in the received signals. For example, smoothing the received signals via a LPF may facilitate elimination of high frequency noise that may appear due to operation of the therapeutic laser beam 220, movement of the distal end 126 of the optical fiber 104, electronic noise, or any other physical phenomena that may affects or contaminate the received signals.

Processing unit 112 can execute instructions 128 to derive a filtered (or smoothed) received signal for each of the sixteen (16) signals received at blocks block 302 and block 306. The filtered received signal can be derived based on any of a variety of smoothing algorithms or equations, such as, for example, a mean filter, a low pass filter, a high pass filter, a bandpass filter, a gaussian filter, a Savitzky-Golay filter, or the like.

Continuing to decision block 310 “signals in acceptable ranges?” a determination can be made as to whether the signals are in acceptable ranges. For example, processing unit 112 can execute instructions 128 to determine whether the signals received at blocks block 302 and block 306 are within a raw signal threshold range of values. From decision block 310, method 300 can continue to block 312 or done block 314. For example, where a determination is made that the received signals are within the raw signal threshold range of values, the method 300 can continue from decision block 310 to block 312. Alternatively, where a determination is made that any one of the received signals are not within the raw signal threshold range of values, the method 300 can continue from decision block 310 to done block 314. At done block 314, method 300 can terminate. In some embodiments, processing unit 112 can execute instructions 128 to generate an error and display the error on a display screen associated with the interrogation sub-system 200.

At block 312 “normalize the received signals” the received signals (e.g., the signals received at block 302 and block 306) can be normalized. For example, processing unit 112 can execute instructions 128 to normalize the received signals based on a period of N last samples. In general, processing unit 112 executes instructions 128 to derive normalized signals to eliminate, from the received signals, bias not related to the measured signals. In general, processing unit 112 executes instructions 128 to derive normalized signals to eliminate, from the received signals, bias not related to the measured signals. For example, processing unit 112 can execute instructions 128 to derive normalized signals for each component of the interrogation laser pulses 118 (e.g., laser beams 204a, 204b, and 204c) based on removing contributions to each signal from the dark current and then normalizing the signals of each the three (3) signal detectors with the signal from the reference detector 408.

Accordingly, the signals are normalized by subtracting the filtered dark current signals of each of the four (4) detectors from each of the other 12 filtered signals respectively. These signals can be referred to as the “unbiased” signals. Subsequently, the unbiased signals for each laser source (e.g., 1310 nm source, 1340 nm source, 1431 nm source, etc.) from the three (3) signal detectors are divided by the respective unbiased signal from the reference detector to compensate for any laser source intensity fluctuations and to eliminate any dependency in laser emitting power. As such, there are nine (9) normalized signals (e.g., one for each component of interrogation laser beams 210 and each of the three (3) signal detector 244a, signal detector 244b, and light snatch detector 248).

Continuing to decision block 316 “integrity check pass?” a determination can be made as to whether the integrity of the optical fiber 104 passes known check. For example, processing unit 112 can execute instructions 128 to determine whether a change over time in the normalized signals from the light snatch detector 248 is greater than a fiber integrity threshold. From decision block 316, method 300 can continue to block 318 or return to done block 314. For example, where a determination is made that the integrity check passed, the method 300 can continue from decision block 316 to block 318. Alternatively, where a determination is made that the integrity check did not pass, the method 300 can return to done block 314 from decision block 316.

At block 318 “derive correct reflected signals from the normalized signals and a distal facet reflection value” corrected reflected power signals can be generated based on the normalized reflected power signals and a distal facet reflection value. As used herein, the distal reflection value is the derived value of the contribution of the distal facet reflection beam 234, which is repeatedly updated as described herein. In general, corrected reflected power signals are derived based on subtracting both the internal reflections and the last known fiber distal end reflection normalized signals from the normalized signals associated with components of the interrogation laser beams 210 having low water absorption coefficients (e.g., the 1310 nm and 1340 nm laser sources, or the like). As such, the resulted signals represent reflections only from the target (e.g., target reflection beam 232) and eliminates internal reflections (e.g., internal reflection beam 238) and reflections from the distal end 126 of the optical fiber 104 (e.g., distal facet reflection beam 234). It is noted that the subtraction is done twice, once without considering the normalized signals associated with the calibration component of the interrogation laser beams 210 (e.g., the 1431 nm laser source, or the like) and once with correcting for the current known distal facet reflection values for the high and low water absorption components of the interrogation laser beams 210.

Further, the correction is conducted using the ratio between the normalized signal of the calibration component signal (e.g., the 1431 nm laser signal) and its last known fiber distal end reflection normalized signals (e.g., the distal facet reflection value). This is to compensate for changes in the reflections from the distal end 126 (e.g., distal facet reflection beam 234) that are represented by the calibration component but not incorporated yet into the last known distal facet reflection values for the high and low absorption components.

With some embodiments, processing unit 112 can execute instructions 128 to derive corrected reflected power signals based on subtracting the product of the distal facet reflection values for each of the high and low water absorption components (e.g., 1340 nm and 1310 nm) and a ratio of the normalized signal for the calibration component (e.g., 1410 nm) and the distal facet reflection values for the calibration component (e.g., 1410 nm) from the normalized signals for the high and low water absorption components (e.g., 1340 nm and 1310 nm), which are based on signals from one or more of the detectors 244a or 244b as outlined above.

Further, processing unit 112 can execute instructions 128 to derive corrected reflected power signals that do not account for the calibration component based on a difference between the normalized signals and the distal facet reflection values.

Accordingly, output from block 318 is four (4) normalized corrected signals associated with the high and low water absorption components (e.g., 1340 nm and 1310 nm) of the interrogation laser beams 210 and arriving at one of the signal detectors (e.g., signal detector 244a or signal detector 244b), two (2) of them considering the calibration component and two (2) without considering the calibration component.

Continuing to decision block 320 “corrected signals less than a threshold value?” a determination is made as to whether the corrected signals derived at block 318 are less than a threshold value. For example, processing unit 112 can execute instructions 128 to determine whether any one of the derived corrected signals are less than a threshold value, such as, corrected signal threshold values. In some embodiments, the corrected signal threshold values can include a value for each of the selected components of the reflected power signals.

Said differently, each one of the corrected signals derived at block 318 is checked to verify if distance should be calculated. For example, if the value of any one of the corrected signals is too low (e.g., less than the threshold), the distance 130 is automatically considered far enough from the target without further calculations. The threshold is either a predefined absolute minimum necessary to derive distance or relative to the last known distal facet reflection value. This is done to avoid distance calculation with very low signals, since these are very sensitive to noise and the calculated results may vary between the two extremes of contact and long distance, although the actual distance is constant. The assumption behind this is that low signals are associated with greater distances (e.g., greater than 4 mm, or the like).

From decision block 320, method 300 can continue to decision block 322 or block 328. For example, where a determination is made that the corrected signals are less than the threshold value, method 300 can continue from decision block 320 to decision block 322; while where a determination is made that the corrected signals are not less than the threshold value, method 300 can continue from decision block 320 to block 328. In some embodiments, only where a determination is made than all of the corrected signals are not less than the threshold value will the method 300 continue from decision block 320 to block 328.

At decision block 322 “distance can be derived?” a determination can be made as to whether the distance can be derived. Said differently, a determination can be made as to whether the expected calculated distance is close enough to the target 106, but not in contact with the target 106. For example, if the expected calculated distance is above a predefined threshold distance, the distance is automatically considered far enough from the target (max distance) without further calculations. This is the verification associated with decision block 320 On the other hand, if the expected calculated distance is below a predefined threshold distance, the distance is automatically considered as contact without further calculations. This is done to avoid negative distance calculations, which are not real.

Processing unit 112 can execute instructions 128 to determine whether the corrected reflected signals are within ranges suitable for deriving the distance 130 based on the following pseudo code:

IF: the distal facet reflection value is less than or equal to the threshold (e.g., product of the threshold and the current distal facet reflection value)

THEN: the distance is the maximum distance

ELSE IF: a product of a ratio of the distal facet reflection values for the high and low water absorption coefficients and DetFactor (e.g., an empirical correction factor to account for differences in the spectral response of the signal detector between the different wavelengths) is greater than or equal to a first signal ratio threshold AND the corrected distal fact reflection value is less than or equal to a the distal facet reflection value threshold

THEN: the distance is the maximum distance

ELSE IF: the product of the ratio of the distal facet reflection values for the high and low water absorption coefficients and DetFactor is less than or equal to a second signal ratio threshold that is less than the first signal ration threshold

THEN: the distance is the maximum distance

ELSE: the distance equals zero (0).

With some examples, the maximum distance for calculation can be 3 mm, 4 mm, 5 mm, 6 mm, 7 mm, 8 mm, or the like).

From decision block 322, method 300 can continue to decision block 324 or block 328. For example, where a determination is made that the distance can be derived, method 300 can continue from decision block 322 to block 328; while where a determination is made that the distance cannot be derived, the method 300 can continue from decision block 322 to decision block 324.

At decision block 324 “normalized received signals stable?” a determination can be made as to whether the normalized received signals are stable. For example, processing unit 112 can execute instructions 128 to determine whether the derived standard deviation of the normalized reflected power signals is less than a stability threshold value. From decision block 324, method 300 can continue to block 326 or return to block 302 (not shown). For example, where a determination is made that the normalized received signals are stable, the method 300 can continue from decision block 324 to block 326; while where a determination is made that the normalized received signals are not stable, the method 300 can continue from decision block 416 to block 302.

At block 326 “update the distal facet reflection value” the distal facet reflection value can be updated. For example, processing unit 112 can execute instructions 128 to derive an updated distal facet reflection value to calibrate the interrogation sub-system 200 as outlined herein. As a specific example, processing unit 112 can execute instructions 128 to update the distal facet reflection value based on the prior measured signal minus the internal reflection value for each component of the reflected signal (e.g., 1310 nm, 1340 nm, 1431 nm, or the like).

At block 328 “derive the distance between a target and the distal end of the optical fiber” a distance between the target and the distal end of the optical fiber can be derived. Said differently, where the estimated distance 130 between the distal end 126 of the optical fiber 104 and the target 106 is not an edge case (e.g., zero (0), Max, etc.) then the distance can be derived according to the fundamental physical equation incorporating the difference between water absorption coefficients of two different wavelengths (e.g., 1340 nm (high) and 1310 nm (low)). This is done twice, once for the distance considering the calibration component (e.g., 1431 nm) and once for the distance not considering the calibration component.

For example, processing unit 112 can execute instructions 128 to derive the distance 130 between the distal end 126 of the optical fiber 104 and the target 106 based on the ratio of the corrected and normalized signals for the high and low water absorption components.

It will further be appreciated that two distances D will be derived. For example, a distance D considering the calibration component (e.g., 1431 nm) and a distance D without considering the calibration component. Processing unit 112 can execute instructions 128 to determine which of the distances to select as the derived distance.

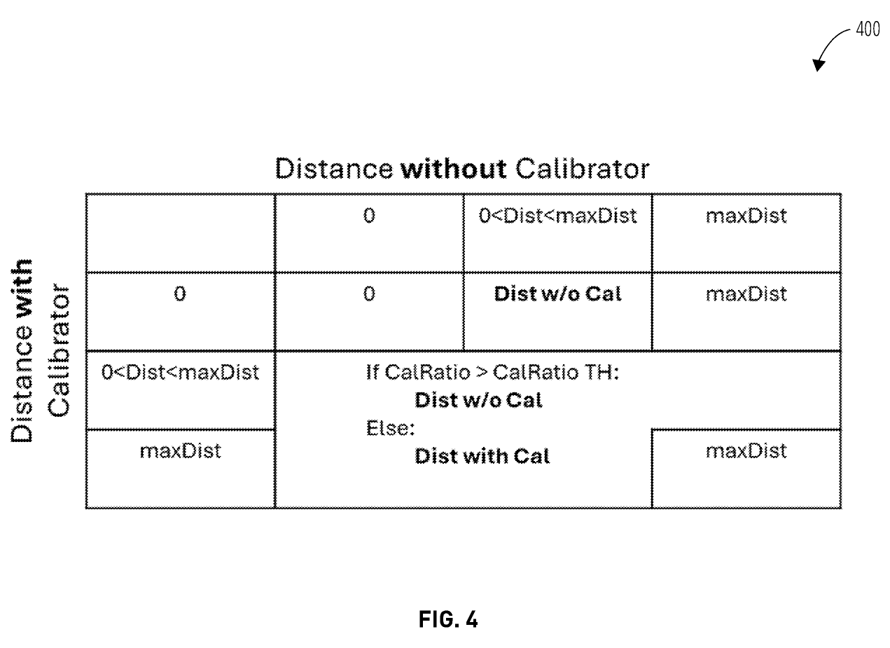

With some embodiments, processing unit 112 can execute instructions 128 to determine which of the derived distances to select based on the table 400 depicted in FIG. 4. Turning now to FIG. 4, the table 400 is depicted. The table 400 is read by finding the cell in which the two derived distances D intersect. More particularly, the upper row corresponds to the distance D (Dist) derived without accounting for the calibration component while the left most column corresponds to the distance D (Dist) derived with accounting for the calibration component.

The cells of the table references maxDist, the variable CalRatio, and CalRatioTH. The reference maxDist is the maximum distance for calculation (e.g., 3 mm, 4 mm, 5 mm, 6, mm, 7 mm, 8 mm, or the like) while CalRatioTH is an empirical threshold related to the variable CalRatio, which can be derived based a ratio of the normalized signals for the calibration component and the distal facet reflection value for the calibration component.

It is to be appreciated that the table 400 can be described as a decision matrix and is based on empirical measurements and statistical analysis on different targets with different reflection properties, different fiber types, used and new fibers, and with different laser procedure treatment modes. The 𝐶𝑎𝑙𝑅𝑎𝑡𝑖𝑜𝑇𝐻 empirical threshold can be fixed for all configurations of the interrogation sub-system 200 and types of the optical fiber 104, different for different types of the optical fiber 104, or dynamically derived during a procedure (e.g., based on properties of the target reflection beam 230 and changes in the distal end 126). Processing unit 112 can execute instructions 128 to determine a single distance D based on the decision matrix in table 400.

Returning to FIG. 3 and the method 300, where the method 300 can continue from block 328 to block 330. At block 330 “down sample and display the distance” the derived and selected distance can be down sampled and displayed for a user. For example, processing unit 112 can execute instructions 128 to remove outlier calculated distance values to avoid sharp changes in the reported distance to the user, which eliminates flickering color changes on the user display. In some embodiments, processing unit 112 can execute instructions 128 to apply a Theil-Sen Estimator (TSE) to remove outliers from the derived and selected distances.

With some embodiments, processing unit 112 can execute instructions 128 to determine a final distance value to report based on the following pseudo code:

INPUT: the last n derived distance samples

loop over all point-pairs [e.g. 1-2, 1-3, 1-4… 2-3, 2-4…] and derive the slope for each point-pair, where the slope is based on a distance measurement and an index and can further be based on a difference between the distance for each point-pair and a different between the index for each point-pair

derive the median of the derived slopes

IF: n is even

THEN: derive the mean of the two median samples

ELSE: the mean is the median sample

loop over all points and derive intercepts based on a sum of the distance and a product of the median slope and the index

derive the median of the intercepts

predict a distance based on a computed fit based on a product of the median sample and the index and the median intercept

verify that the predicted distance does not violate sample bounds (is within minimum and maximum distances

From the above pseudo code, processing unit 112 can determine a single distance value, which can be displayed to a user. In some embodiments, processing unit 112 can execute instructions 128 to display the numerical value of the derived distance while in other embodiments, the derived distance can be converted to a graphical representation (e.g., geometric shape, colored indicator, or the like).

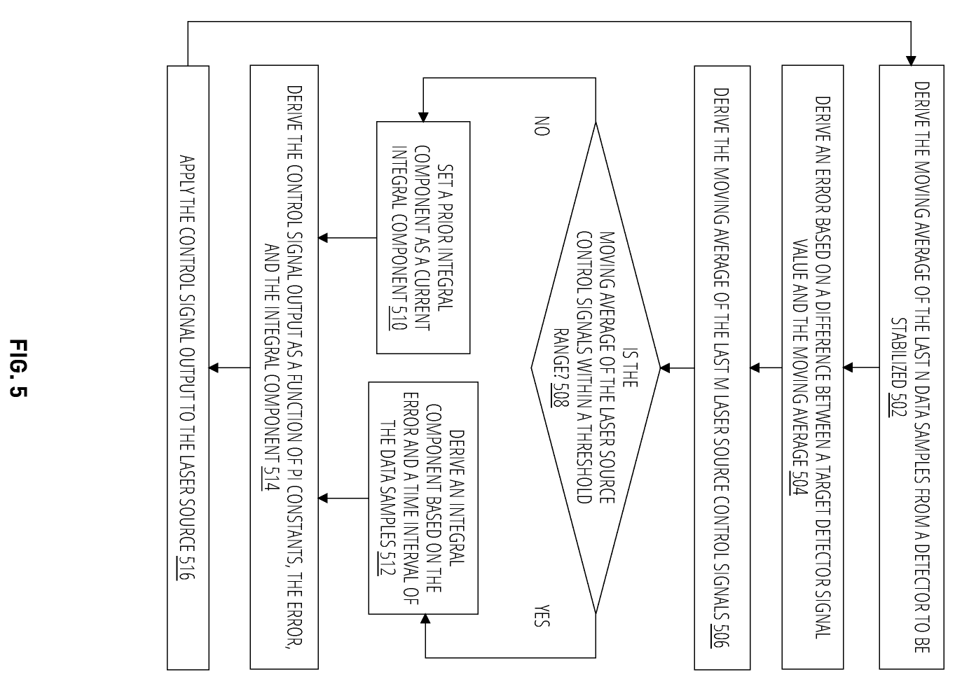

FIG. 5 illustrates a flowchart showing a method 500 for controlling the current gain on a laser source to maintain stable signal levels at a detector. The method 500 is described with reference to the surgical laser system 100 of FIG. 1A and to the interrogation sub-system 200 of FIG. 2. It is to be appreciated however, that the method 500 could be implemented by a surgical system different than the one depicted herein. Further, it is noted that the method 500 can be implemented by the interrogation sub-system 200, and particularly the laser source gain controller 250 of interrogation sub-system 200, for each laser source and detector to be controlled. For example, the interrogation sub-system 200 includes three laser sources 202a, 202b, and 202c. As such, the method 500 could be implemented separately for each laser source. In some embodiments, the method 300 can implement the method 500 at block 304.

The method 500 can begin at block 502. At block 502 “derive the moving average of the last N data samples from a detector to be stabilized” a moving average of the last N data samples from the detector to be stabilized can be derived. For example, processing unit 112 can execute instructions 126 to collect the last N (e.g., 2, 4, 6, 8, 12, 16, 24, 36, etc.) data samples from the reference detector 210 and derive the average to form a moving average (MA_detector). In some examples, processing unit 112 can execute instructions 128 to receive from a detector (e.g., the reference detector 212, or the like) an indication of a power of a laser beam emitted by a laser source (e.g., the laser beam 204a emitted by the laser source 202a, or the like) and can derive a moving average of the received power over N number of samples.

Continuing to block 504 “derive an error based on a difference between a target detector signal value and the moving average” an error for the Nth data sample can be derived based on a difference between a target detector signal value and the moving average can be derived. For example, processing unit 112 can execute instructions 128 to derive an error (Error) based on the difference between a target value and the MA_detector.

Continuing to block 506 “derive the moving average of the last M laser source control signals” a moving average of the last M control signals (e.g., current gain signals, or the like) for the laser source can be derived. For example, processing unit 112 can execute instructions 128 to collect the last M (e.g., 2, 4, 6, 8, 12, 16, 24, 36, etc.) control signals sent to the laser source (e.g., laser source 202a, 202b, or 202c) and derive the average to form a moving average (MA_source). With some examples, MA_source can be limited to a minimum or maximum current gain.

Continuing to decision block 508 “is the moving average of the laser source control signals within a threshold range?” a determination is made as to whether the moving average of the laser source control signals is within a threshold range. For example, each laser source (e.g., the laser sources 202a, 202b, and 202c) can have an associated minimum and maximum current gain. At decision block 508 a determination can be made as to whether the moving average of the current gain control signals is between the minimum and maximum current gains. For example, processing unit 112 can execute instructions 128 to determine whether the MA_source is greater than a minimum current gain threshold or less than a maximum current gain threshold.

From decision block 508, method 500 can continue to block 510 or block 512. For example, where a determination is made that the moving average of the current gain control signals is not between the minimum and maximum current gains, method 500 can continue from decision block 508 to block 510. Alternatively, where a determination is made that the moving average of the current gain control signals is between the minimum and maximum current gains, method 500 can continue from decision block 508 to block 512.

At block 510 “set a prior integral component as a current integral component” a prior integral component can be set at a current integral component. For example, processing unit 112 can execute instructions 128 to set the prior integral component (e.g., i) as the current integral component (e.g., i +1).

At block 512 “derive an integral component based on the error and a time interval of the data samples” an integral component can be derived based on the error and a time interval of the data samples from the detector (e.g., reference detector 212). For example, processing unit 112 can execute instructions 128 to derive an integral component based on the product of the Error and the time interval (dt) plus the prior integral component (e.g., Integralᵢ₊₁ = Integralᵢ + (Error * dt)).

The method 500 can continue from block 510 and/or block 512 to block 514. At block 514 “derive the control signal output as a function of PI constants, the error, and the integral component” a laser source control signal can be derived as a function of PI constants (e.., proportional constant Kp and integral constant Ki), the error, and the integral component. For example, processing unit 112 can execute instructions 128 to derive a laser source current gain control signal (Control Signal) as the sum of the product of Kp and the Error and the product of Ki and the Integral (e.g., Control Signal = (Kp * Error) + (Ki * Integral)).

Continuing to block 516 “apply the control signal output to the laser source” the control signal can be applied to the laser source. For example, processing unit 112 can execute instructions 128 to apply the control signal generated at block 514 to the laser source to be stabilized (e.g., laser source 202a, 202b, or 202c).

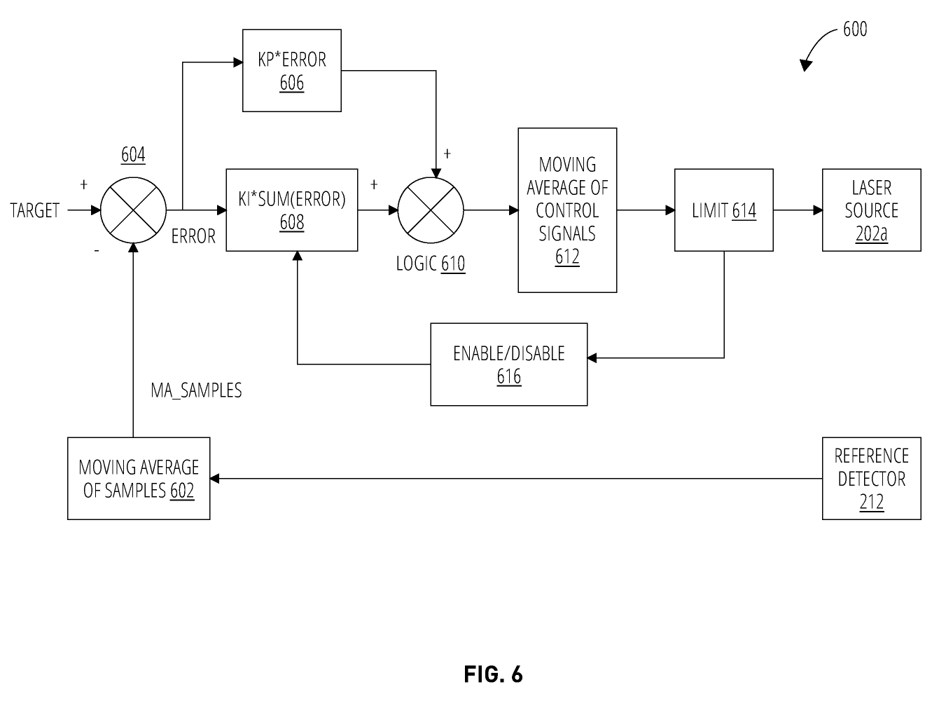

FIG. 6 illustrates, in block form, an example dataflow diagram 600. The dataflow diagram 600 can be configured to control the current gain of a laser source to stabilize signals at a detector as outlined herein. Dataflow diagram 600 could be implemented as part of laser source gain controller 250, or the like. Dataflow diagram 800 refers to a number of data structures, which can be stored in memory 114 and accessed by processing unit 112 in executing instructions 128. As a specific example, dataflow diagram 600 is depicted and described configured to control laser source 202a to stabilize signals on reference detector 212.

dataflow diagram 600 can include logic 602 configured to derive the moving average of N number of samples receive from reference detector 212. dataflow diagram 600 can also include logic 604 configured to derive an error (Error) as the difference between the moving average and a target signal level (Target). dataflow diagram 600 can also include logic 606 and logic 608 configured to derive the product of the error and a proportional constant and to integrate the error and an integral constant.

dataflow diagram 600 can also include logic 610 configured to sum the outputs from the logic 606 and logic 608. dataflow diagram 600 further includes logic 612 configured to derive the moving average of M number of outputs of logic 610 and includes logic 614 configured to constrain the logic 612 to a range (e.g., minimum and maximum current gain, or the like). Further, dataflow diagram 600 includes logic 616 configured to enable or disable integration. For example, where the output from the logic 612 corresponding to the current gain is at the limits (e.g., minimum or maximum), the dataflow diagram 600 is configured to stop integrating and to maintain the integral value from the prior time step. For example, where the current gain control signal output is saturated at the high limit and at the same time, the signal detector value is below the target value, integration will be stopped. As another example, wherein the current gain control signal output is saturated at the low limit and at the same time, the signal detector value is above the target value, the integration will be stopped.

It is noted that although the dataflow diagram 600 is configured to control a laser source relative to stabilize signals at a single detector (e.g., reference detector 212, signal detectors 244a, and/or signal detector 244b) it is important to ensure that the other detectors are not saturated. As such, with some examples, the maximum current gain is set at levels that will not saturate the other detectors.

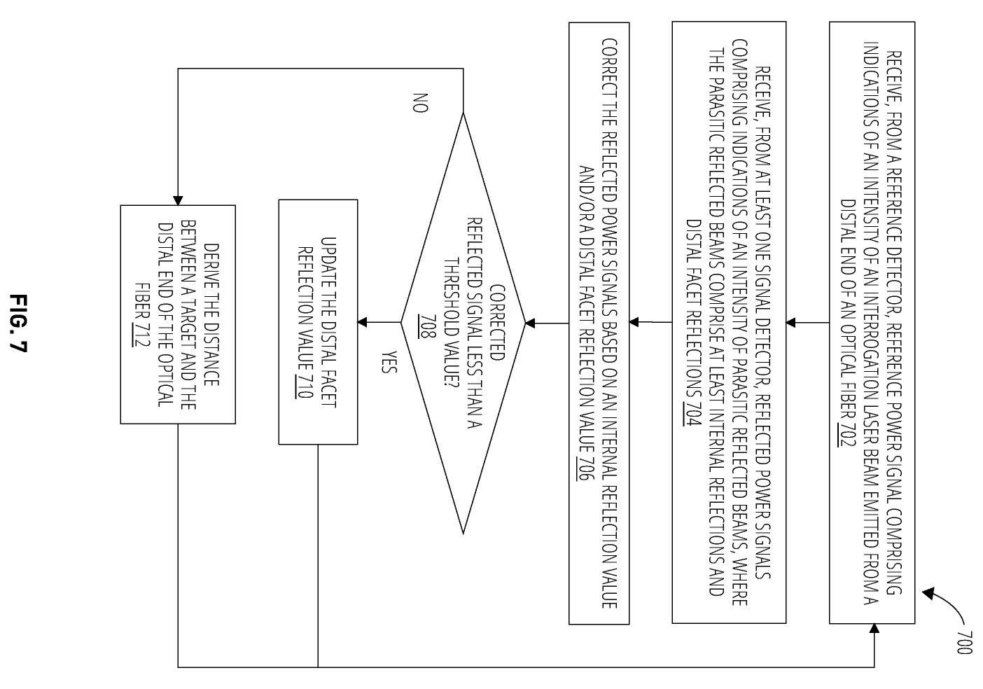

FIG. 7 illustrates a flowchart showing a method 700 for calibrating a distance measurement system. The method 700 is described with reference to the surgical laser system 100 of FIG. 1A and to the interrogation sub-system 200 of FIG. 2. It is to be appreciated however, that the method 700 could be implemented by a surgical system different than the one depicted herein. Further, the method 700 is described with reference to the method 300 of FIG. 3 and could be implemented as part of the distance measurement techniques described herein.

The method 700 can begin at block 702. At block 702 “receive, from a reference detector, reference power signal comprising indications of an intensity of an interrogation laser beam emitted from a distal end of an optical fiber” a reference power signal comprising indications of an intensity of an interrogation laser beam emitted from a distal end of an optical fiber can be received. For example, processing unit 112 can execute instructions 128 to receive from reference detector 212, signals comprising indications of the power of interrogation laser beams 208. As noted above, interrogation laser beams 210 can comprise multiple components (e.g., laser beams 204a, 204b, 204c, etc.) In some examples, processing unit 112 can execute instructions 128 to receive indications of a power of each respective component.

Continuing to block 704 “receive, from at least one signal detector, reflected power signals comprising indications of an intensity of parasitic reflected beams, where the parasitic reflected beams comprise at least internal reflections and distal facet reflections” reflected power signals comprising indications of an intensity of parasitic reflected beams can be received, where the parasitic reflected beams comprise at least internal reflections and distal facet reflections. For example, processing unit 112 can execute instructions 128 to receive from signal detectors 244a and/or 244b, signals comprising indications of the power of parasitic reflected beams 240. As noted above, parasitic reflected beams 240 can comprise multiple components (e.g., distal facet reflection beam 234, proximal facet reflection beam 236, internal reflection beam 238, etc.) In some examples, proximal facet reflection beams 236 can be optically eliminated from the parasitic reflected beams 240 (e.g., by light snatch mirror 246 and light snatch detector 248, or the like). As such, signals generated by signal detectors 244aand 244b will depend only on distal facet reflection beam 234 and internal reflection beam 238. Further, as introduced above there will be periods of time during a procedure where the target 106 is far enough away from the distal end 126 of the optical fiber 104 that all of interrogation laser beams 210 will be absorbed by the liquid medium, and as such, target reflection beam 232 will be substantially zero, or will be small enough to allow calibration as described herein.

Continuing to block 706 “correct the reflected power signals based on an internal reflection value and/or a distal facet reflection value” the reflected power signals can be corrected based on an internal reflection value and a distal facet reflection value. As used herein, the internal reflection value is the known value of the contribution of the internal reflection beam 238; while the distal reflection value is the derived value of the contribution of the distal facet reflection beam 234, which is repeatedly updated as described herein.

With some embodiments, processing unit 112 can execute instructions 128 to derive the corrected reflected power signals as the difference between the reflected power signals and the internal reflection value and the distal facet reflection value. Said differently, processing unit 112 can execute instructions 128 to subtract the internal reflection value and the distal facet reflection value from each component (or selected components) of the reflected power signals.

In some embodiments, processing unit 112 can execute instructions 128 to derive the corrected reflected power signals as the difference between the reflected power signals and the internal reflection value only. Said differently, processing unit 112 can execute instructions 128 to subtract the internal reflection value from each component (or selected components) of the reflected power signals.

As noted, processing unit 112 can execute instructions 128 to derive corrected reflected power signals for select components. For example, described above, the interrogation laser beams 210 may have multiple components (e.g., laser beams 204a, 204b, and 204c). As a specific example, laser beams 204a, 204b, and 204c may have approximate wavelengths of 1310 nm, 1340 nm, and 1431 nm. In such an example, processing unit 112 can execute instructions 128 to derive corrected reflected power signals for the 1310 nm and 1340 nm components of the signals.

Continuing to decision block 708 “corrected reflected signals less than a threshold value?” a determination is made as to whether the corrected reflected signals are less than a threshold value. For example, processing unit 112 can execute instructions 128 to determine whether the corrected reflected signals are less than a threshold value, such as, a corrected signal threshold value. In some embodiments, the corrected signal threshold value can include a value for each component of the reflected power signals, or for the selected components for which corrected reflected power signals are derived. With some embodiments (e.g., where the corrected reflected power signal is based on both the internal reflection value and the distal facet reflection value) the corrected signal threshold values can be a predefined absolute threshold. With other examples (e.g., where the corrected reflected power signal is based on only the internal reflection value) the corrected signal threshold values can be based on the most recently derived distal facet reflection value). For example, in some embodiments, the threshold can be less than or equal to 5% of the prior distal facet reflection value, less than or equal to 3% of the prior distal facet reflection value, or less than or equal to 1.5% of the prior distal facet reflection value.

From decision block 708, method 700 can continue to block 710 or block 712. For example, where a determination is made that the corrected reflected signals are less than the threshold value, method 700 can continue from decision block 708 to block 710. In some embodiments, where a determination is made than any one component of the corrected reflected signals (e.g., 1310 nm component, 1340 nm component, or the like) is less than the threshold value then the method 700 can continue from decision block 708 to block 710.

Alternatively, where a determination is made that the corrected reflected signals are not less than the threshold value, method 700 can continue from decision block 708 to block 712. In some embodiments, only where a determination is made than all of the selected components of the corrected reflected signals (e.g., 1310 nm component and 1340 nm component, or the like) are not less than the threshold value will the method 700 continue from decision block 708 to block 712.

At block 710 “update the distal facet reflection value” the distal facet reflection value can be updated. For example, processing unit 112 can execute instructions 128 to derive an updated distal facet reflection value to calibrate the interrogation sub-system 200 as outlined herein. As a specific example, processing unit 112 can execute instructions 128 to update the distal facet reflection value based on the prior measured signal minus the internal reflection value for each component of the reflected signal (e.g., 1310 nm, 1340 nm, 1431 nm, or the like).

At block 712 “derive the distance between a target and the distal end of the optical fiber” a distance between the target and the distal end of the optical fiber can be derived. For example, processing unit 112 can execute instructions 128 to derive the distance 130 between the distal end 126 of the optical fiber 104 and the target 106. In general, the distal can be derived based on a ratio of the target reflection beam 232 and the interrogation laser beams 210. Said differently, processing unit 112 can execute instructions 128 to derive the distance 130 based on a ratio of the output from the reference detector 212 and the signal detectors 244a and 244b where the output from the signal detectors 244a and 244b are corrected based on the internal reflection value and the distal facet reflection value to remove contributions of distal facet reflection beam 234 and internal reflection beam 238 from the signals.

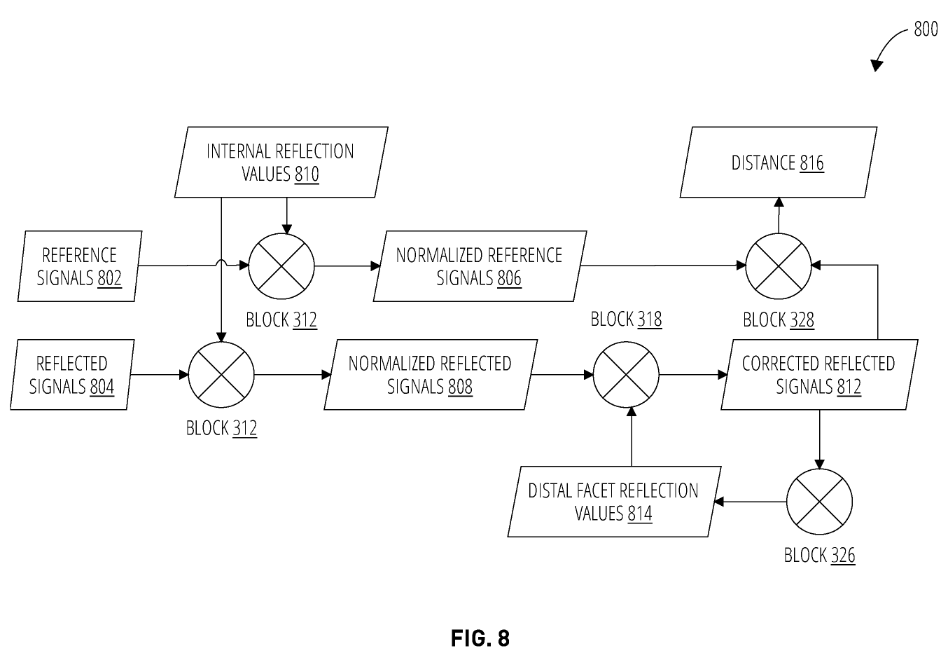

FIG. 8 illustrates, in block form, an example dataflow diagram 800 showing signals and threshold values described herein as part of a distance measurement process and/or a calibration process for a distance measurement system. Dataflow diagram 800 refers to a number of data structures, which can be stored in memory 114 and accessed by processing unit 112 in executing instructions 128. Further, dataflow diagram 800 refers to the interrogation sub-system 200 of FIG. 2 as well as steps or operations of method 300 of FIG. 3 for convenience in describing the diagram.

The dataflow diagram 800 can begin with reference signals 802 and reflected signals 804. In general, reference signals 802 can correspond to the signals for each component of interrogation laser beams 210 received from reference detector 212 while reflected signals 804 can correspond to signals for each component of interrogation laser beams 210 received from signal detectors 244a and 244b, which can correspond to target reflection beam 232, distal facet reflection beam 234, and/or internal reflection beam 238.

At block 312, normalized reference signals 806 and normalized reflected signals 808 are derived (e.g., by processing unit 112 executing instructions 128, or the like). For example, normalized reference signals 806 and normalized reflected signals 808 can be derived by subtracting internal reflection values 810 from reference signals 802 and reflected signals 804 as described above.

At block 318, corrected reflected signals 812 are derived (e.g., by processing unit 112 executing instructions 128, or the like). For example, corrected reflected signals 812 can be derived by subtracting distal facet reflection values 814 from normalized reflected signals 808. Given corrected reflected signals 812, either distal facet reflection values 814 can be updated or the distance 816 (e.g., the distance 130, or the like) can be derived. For example, where the distance 130 between the distal end 126 of the optical fiber 104 and the target 106 is greater than a threshold (e.g., 4 mm, 6 mm, 8 mm, or the like) distal facet reflection values 814 can be updated based on an average of N prior corrected reflected signals 812. Alternatively, where the distance 130 between the distal end 126 of the optical fiber 104 and the target 106 is less than the threshold the distance 816 can be derived.

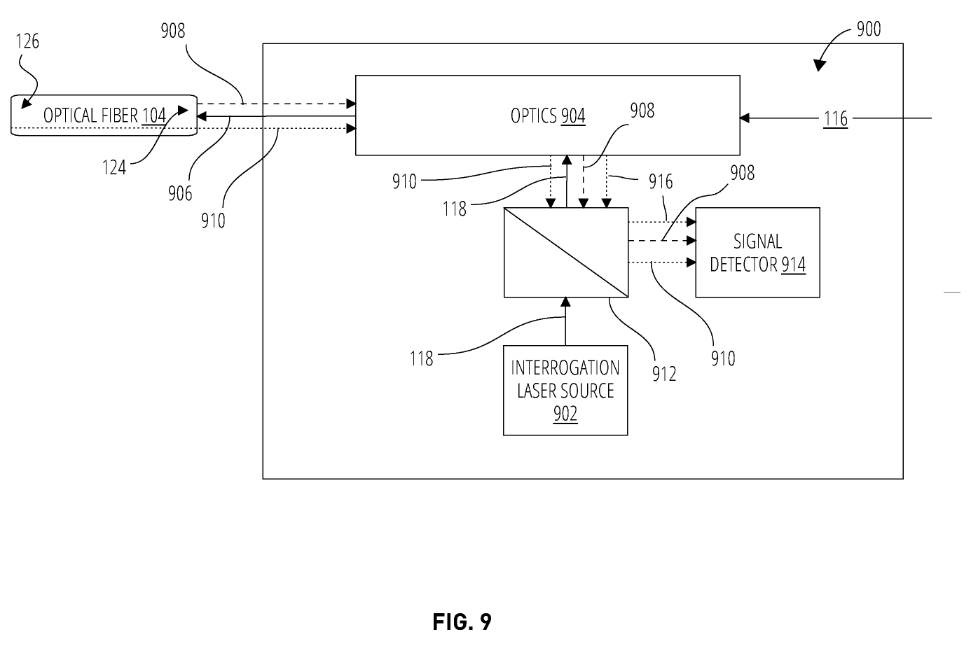

FIG. 9 illustrates an example interrogation sub-system 900, which can be provided in accordance with some embodiments of the present and used to interpolate optical fiber damage. For example, the interrogation sub-system 900 can be provided as the interrogation sub-system 110 of the surgical laser console 102 shown as part of the surgical laser system 100 in FIGS. 1A and 1B .

Interrogation sub-system 900 can include an interrogation laser source 902 and optics 904. The interrogation laser source 902 can be configured to generate one or more pulsed laser beams, which can form the interrogation laser pulses 118. With some embodiments (e.g., see FIG. 2), interrogation laser source 902 can be configured to generate interrogation laser pulses 118 having multiple wavelength components.

Optics 904 can be configured to receive the interrogation laser pulses 118 from the interrogation laser source 902 and receive therapeutic laser pulses 116 from a therapeutic laser source (e.g., the therapeutic laser source 108, or the like). The optics 904 can further be configured to combine the therapeutic laser pulses 116 and interrogation laser pulses 118 to form combined laser beam 906 and couple the combined laser beam 906 to the optical fiber 104, such that the combined laser beam 906 can be delivered to the target 106 as described above. The optics 904 can include any of a variety of optical component (e.g., multiplexors, polarizers, beam combiners, beam splitters, collimators, circulators, lenses, etc.) arranged to condition and direct the therapeutic laser pulses 116 and the interrogation laser pulses 118 (e.g., the combined laser beam 906) to the optical fiber 104.

As described above, during operation, some portion of the combined laser beam 906 will be reflected by the proximal end 124 as proximal facet reflection 908 (e.g., proximal facet reflection beam 236) and some portion of the combined laser beam 906 will be reflected by the distal end 126 as distal facet reflection 910 (e.g., distal facet reflection beam 234). As noted, where the the combined laser beam 906 includes multiple wavelength components, the proximal facet reflection 908 and distal facet reflection 910 may also include multiple wavelength components. As outlined above, during operation, the proximal end 124 and distal end 126 of the optical fiber 104 will degrade and/or be damaged due to the energy of the combined laser beam 906. More particularly, energy from the therapeutic laser pulses 116, which is part of the combined laser beam 206, will cause damage to the optical fiber 104. The degradation and/or damage of the optical fiber 104 can be interpolated based on the change in the proximal facet reflection 908 and distal facet reflection 910 over time. To that end, the interrogation sub-system 200 further includes a beam splitter 912 and signal detector 914, which can be configured to measure the proximal facet reflection 908 and distal facet reflection 910.

With some embodiments, the beam splitter 912 is disposed between the interrogation laser source 902 and the optics 904. In this arrangement, the beam splitter 912 can be configured to direct the interrogation laser pulses 118 to the optics 904 and direct the proximal facet reflection 908 and distal facet reflection 910 to the signal detector 914. The signal detector 914 can be any of a variety of light detectors. In general, such light detectors may include devices that detect and/or measure characteristics of light beams and encode the detected and/or measured characteristics in electrical signals. Accordingly, the signal detector 914 is configured to measure an intensity of the proximal facet reflection 908 and distal facet reflection 910.

Processing unit 112 can be coupled to signal detector 914 and can execute instructions 128 to receive signals comprising indications of the intensity of proximal facet reflection 908 and distal facet reflection 910. Further, processing unit 112 can execute instructions 128 to interpolate damage to the optical fiber 104. It is to be appreciated, that interrogation sub-system 900 is configured to measure an intensity of the reflected light due to both the proximal facet reflection 908 and the distal facet reflection 910. It is noted that there is not a reflection from the target 106 depicted in this figure. It is to be appreciated that during a treatment procedure, there will be many time periods, even short ones, where the target 106 is far enough away (e.g., greater than 4 millimeters (mm), greater than 6 mm, or the like) from the distal end 126 of the optical fiber 104 that any reflection from the target 106 will be insignificant and/or immeasurable. Further, there will be reflections from the optical fiber 104, which can be referred to as internal reflections 916. However, these reflections will remain constant throughout a procedure as the optical fiber 104 should not be damaged and/or degrade during the procedure.

As such, with some embodiments, processing unit 112 can execute instructions 128 to measure the distance 130 and to measure the intensity of proximal facet reflection 908 and distal facet reflection 910 during periods where the distance 130 is greater than a threshold distance (e.g., 4 mm, 6 mm, 8 mm, or the like). During these periods, the output from the signal detector 914 represents only proximal facet reflection 908 and distal facet reflection 910 (when corrected for internal reflections 916). Further, the processing unit 112 can execute instructions 128 control the surgical laser system 100 based on the signals from the signal detector 914. This is described in greater detail below. However, in general, as the optical fiber 104 degrades and/or is damaged during a procedure, the intensity of the proximal facet reflection 908 and distal facet reflection 910 will decrease. Processing unit 112 can execute instructions 128 to interpolate the amount of damage or degradation the optical fiber 104 has experienced and can control and/or suggest actions based on the interpolated degradation and/or damage.

With some embodiments, the intensity of the proximal facet reflection 908 and distal facet reflection 910 can be measured independently from each other (e.g., as described with reference to FIG. 2).

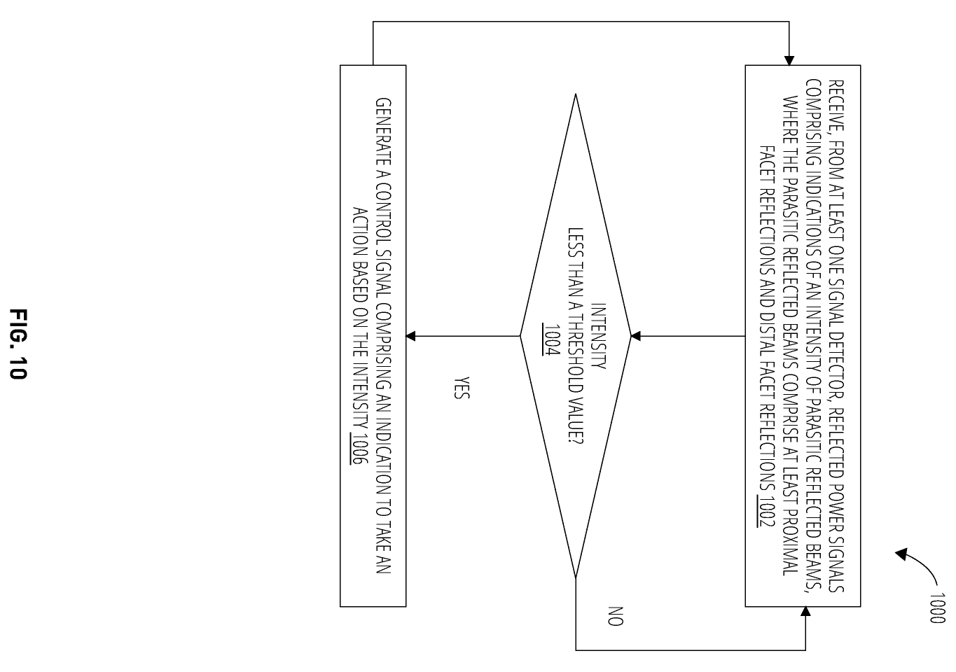

FIGS. 10, 11, 12, and 13, illustrate, respectively, example methods 1000, 1100, 1200, and 1300 for verifying the integrity of an optical fiber and/or controlling a surgical laser console based in interpolated damage to the optical fiber, according to at least one embodiment of the disclosure. With some embodiments, methods 1000, 1100, 1200, or 1300 can be implemented by the methods method 300 of FIG. 3 at decision block 316. Further, the methods 1000, 1100, 1200, and/or 1300 be implemented during a surgical laser procedure, such as a laser lithotripsy procedure and are described herein with respect to the surgical laser system 100 of FIGS. 1A and 1B and the interrogation sub-system 200 of FIG. 2 or interrogation sub-system 900 of FIG. 9. It is to be appreciated however, that the methods 1000, 1100, 1200, and/or 1300 could be implemented by a surgical system different than the one depicted herein.

Turning to FIG. 10 and the method 1000. The method 1000 can begin at block 1002. At block 1002 “receive, from at least one signal detector, reflected power signals comprising indications of an intensity of parasitic reflected beams, where the parasitic reflected beams comprise at least proximal facet reflections and distal facet reflections” reflected power signals comprising indications of an intensity of parasitic reflected beams can be received, where the parasitic reflected beams comprise at least proximal facet reflections and distal facet reflections. For example, processing unit 112 can execute instructions 128 to receive from signal detector 914, signals comprising indications of the intensity (or power) of at least proximal facet reflection 908 and distal facet reflection 910. In other embodiments, processing unit 112 can execute instructions 128 to receive, signals comprising indications of the intensity of proximal facet reflection beam 236 from signal light snatch detector 248 and signals comprising indications of the intensity of distal facet reflection beam 234 from the signal detectors 244a and/or 244b. As noted above, in some embodiments, signals comprising indications of the distal facet reflection 210 will include indications of other parasitic reflections (e.g., internal reflection beam 238, or the like). Further, with some embodiments, multiple signal measurements (e.g., a time series of signals, or the like) from the detectors can be received.

Further, as introduced above there will be periods of time during a procedure where the target 106 is far enough away from the distal end 126 of the optical fiber 104 that all of interrogation laser pulses 118 will be absorbed by the liquid medium, and as such, target reflected laser pulses 120 will be substantially zero, or will be small enough such that the reflected light incident on the signal detectors 244a and 244b will only be representative of the distal facet reflection beam 234 and internal reflection beam 238. With some embodiments, processing unit 112 can execute instructions 128 to receive signals from the signal detectors (e.g., signal detectors 244a, 244b, and/or signal detector 914) during periods where the target 106 is far enough away such that the received signals are only dependent on parasitic reflections as outlined above. Further, as outlined above, in some embodiments the reflections can be based on an incident laser beam having multiple wavelength components (e.g., laser beams 204a, 204b, 204c, etc.). As such, signals received from the signal detectors can correspond to these multiple wavelength components.

Additionally, with some embodiments, processing unit 112 can execute instructions 128 to receive signals from signal detectors comprising indications of “dark current." For example, where the therapeutic laser pulses 116 and interrogation laser pulses 118 are off, the detectors (e.g., signal detector 914, signal detectors 244a, 244b, etc.) will measure some light intensity, which is referred to herein as “dark current” intensity. Processing unit 112 can execute instructions 128 to measure this dark current and interpolate damage to the optical fiber 104 based on the intensity of the dark current.

Continuing to decision block 1004 “intensity less than a threshold value?” a determination can be made as to whether the intensity (e.g., intensity of the reflected signals) is less than a threshold value. For example, processing unit 112 can execute instructions 128 to determine whether the intensity of the signals received from the signal detectors are less than a threshold value. With some embodiments, the threshold value can be preset. With other examples, processing unit 112 can execute instructions 128 to determine the threshold at the start of each procedure when the optical fiber 104 is new and before it has been exposed to therapeutic laser pulses 116. For example, processing unit 112 can execute instructions 128 to measure the parasitic reflections (e.g., proximal facet reflection 908, distal facet reflection 910, internal reflection beam 238, etc.) and determine a baseline level of the reflections. Further, processing unit 112 can execute instructions 128 to set the threshold based on this baseline value, such as, for example by setting the threshold as the baseline multiple by a constant value. For example, for the proximal facet reflection 908 and/or distal facet reflection 910, processing unit 112 can execute instructions 128 to set the threshold as the baseline value multiplied by a constant less than 1 (e.g., 0.9, 0.8, 0.7, 0.6, 0.5, etc.) As another example, for the dark current value, processing unit 112 can execute instructions 128 to set the threshold as the baseline value multiplied by a constant greater than 1 (e.g., 1.25, 1.5, 1.75, 2.0, 3.0, 4.0, etc.)

From decision block 1004, method 1000 can continue to block 1006 or return to block 1002. For example, where a determination is made that the intensity is less than the threshold value, the method 1000 can continue from decision block 1004 to block 1006 while the method 1000 can return to block 1002 from decision block 1004 where a determination is made that the intensity is not less than the threshold value.

At block 1006 “generate a control signal comprising an indication to take an action based on the intensity” a control signal comprising an indication to take an action based on the intensity can be generated. For example, processing unit 112 can execute instructions 128 to generate a control signal comprising an indication for the surgical laser system 100 to take some action based on the intensity. In some embodiments, processing unit 112 can execute instructions 128 to generate a control signal to cause a change in operation of the surgical laser system 100 (e.g., increase pulse frequency of therapeutic laser pulses 116, increase pulse energy of therapeutic laser pulses 116, increase pulse frequency and energy of therapeutic laser pulses 116, change a suggested working distance based on measuring distance 130, or stop generation of the therapeutic laser pulses 116) to compensate for the degradation of the optical fiber 104 or to prevent damage to the surgical laser system 100. With other embodiments, processing unit 112 can execute instructions 128 to generate a control signal to cause the surgical laser system 100 to display an alert to the user (e.g., an alert suggesting a change in operating parameters such as pulse frequency and/or energy, an alert suggesting the user cleave the distal end 126 of the optical fiber 104 to renew the optical fiber 104, or an alert indicating a reduction is lasing efficiency due to degraded optical fiber 104).

For example, a large (e.g., 40%, 50%, 60%, or the like) decrease in the intensity of the proximal facet reflection 908 from the baseline can indicate severe damage to the proximal end 124 of the optical fiber 104, which can be indicative of a “dead” optical fiber that does not transmit enough laser energy. Continued lasing with such an optical fiber is not efficient and may result in damage to the surgical laser system 100 (e.g., damage to the port 228, damage to a blast shield, or the like). As another example, a small (e.g., 5%, 10%, 15%, 20%, 25%, or the like) decrease in the intensity of the proximal facet reflection 908 and/or distal facet reflection 910 from the baseline can indicate damage to the proximal end 124 and/or distal end 126 of the optical fiber 104, which may result in a corresponding decrease in the energy of the therapeutic laser pulses 116 delivered from the optical fiber 104. As such, adjusting the operational parameters of the surgical laser system 100 (e.g., corresponding increase in pulse frequency and/or energy of the therapeutic laser pulses 116) may result in a consistent delivery of energy of therapeutic laser pulses 116 ultimately delivered from the optical fiber 104.

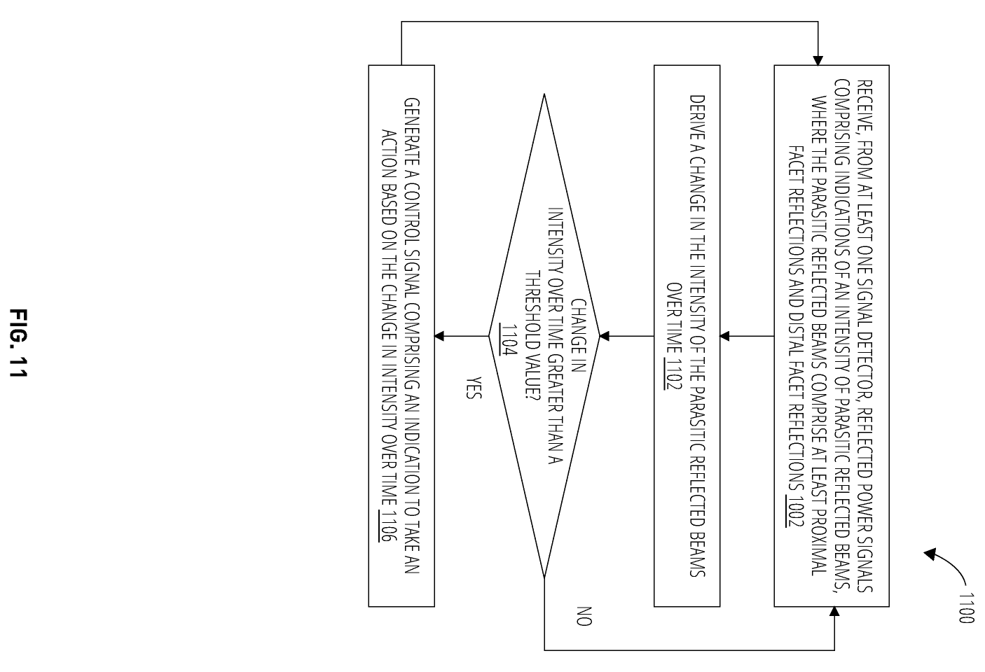

Turning now to FIG. 11 and the method 1100, which is further described with reference to method 1000 of FIG. 10. The method 1100 can begin at block 1002 and continue to block 1102. At block 1102 “derive a change in the intensity of the parasitic reflected beams over time” an intensity over time of the parasitic reflected beams can be determined. For example, processing unit 112 can execute instructions 128 to determine an intensity of the proximal facet reflection 908, distal facet reflection 910 and/or dark current reflections over a time period (e.g., hundreds of seconds, minutes, or the like). As a specific example, processing unit 112 can execute instructions 128 to determine a change in of the intensity of the proximal facet reflection 908 and/or distal facet reflection 910 over a time period of 120 seconds, 240 seconds, 360 seconds, between 120 and 600 seconds, or the like. With some embodiments, processing unit 112 can execute instructions 128 to determine a change in the intensity of the proximal facet reflection 908 and/or distal facet reflection 910 over time and to further derive a slope, or rate of the change, over the time period.

Continuing to decision block 1104 “change in intensity over time greater than a threshold value?” a determination can be made as to whether the change in intensity over time is greater than a threshold value. For example, processing unit 112 can execute instructions 128 to determine whether the change in the intensity of the proximal facet reflection 908 and/or distal facet reflection 910 over time is greater than a threshold value. Further, where at block 1102 the slope or rate of chance is derived, processing unit 112 can execute instructions 128 to determine whether the slope is greater than a threshold value. With some embodiments, the threshold values can be preset.

From decision block 1104, method 1100 can continue to block 1106 or return to block 1002. For example, where a determination is made that the change in intensity over time (or the slope of the change) is greater than the threshold value the method 1100 can continue from decision block 1104 to block 1106 while the method 1100 can return to block 1002 from decision block 1104 where a determination is made that the change in intensity over time (or the slope of the change) is not greater than the threshold value.

At block 1106 “generate a control signal comprising an indication to take an action based on the change in intensity over time” a control signal comprising an indication to take an action based on the change in intensity over time can be generated. For example, processing unit 112 can execute instructions 128 to generate a control signal comprising an indication for the surgical laser system 100 to take some action based on the change in intensity (or the slope of the change). In some embodiments, processing unit 112 can execute instructions 128 to generate a control signal to cause a change in operation of the surgical laser system 100 (e.g., increase pulse frequency of therapeutic laser pulses 116, increase pulse energy of therapeutic laser pulses 116, increase pulse frequency and energy of therapeutic laser pulses 116, change a suggested working distance based on measuring distance 130, or stop generation of the therapeutic laser pulses 116) to compensate for the degradation of the optical fiber 104 or to prevent damage to the surgical laser system 100. With other embodiments, processing unit 112 can execute instructions 128 to generate a control signal to cause the surgical laser system 100 to display an alert to the user (e.g., an alert suggesting a change in operating parameters such as pulse frequency and/or energy, an alert suggesting the user cleave the distal end 126 of the optical fiber 104 to renew the optical fiber 104, or an alert indicating a reduction is lasing efficiency due to degraded optical fiber 104).

For example, a large (e.g., 40%, 50%, 60%, or the like) decrease in the intensity of the proximal facet reflection 208 from the baseline can indicate severe damage to the proximal end 124 of the optical fiber 104, which can be indicative of a “dead” optical fiber that does not transmit enough laser energy. Continued lasing with such an optical fiber is not efficient and may result in damage to the surgical laser system 100 (e.g., damage to the port 228, damage to a blast shield, or the like). As another example, a small (e.g., 5%, 10%, 15%, 20%, 25%, or the like) decrease in the intensity of the proximal facet reflection 908 and/or distal facet reflection 910 from the baseline can indicate damage to the proximal end 124 and/or distal end 126 of the optical fiber 104, which may result in a corresponding decrease in the energy of the therapeutic laser pulses 116 delivered from the optical fiber 104. As such, adjusting the operational parameters of the surgical laser system 100 (e.g., corresponding increase in pulse frequency and/or energy of the therapeutic laser pulses 116) may result in a consistent delivery of energy of therapeutic laser pulses 116 ultimately delivered from the optical fiber 104. As another example, where the dark current reading increases above the baseline for a long enough time, it may indicate that the distal end 126 of the optical fiber 104 is burnt and should be cleaved to maintain transmission efficiency of the optical fiber 104.

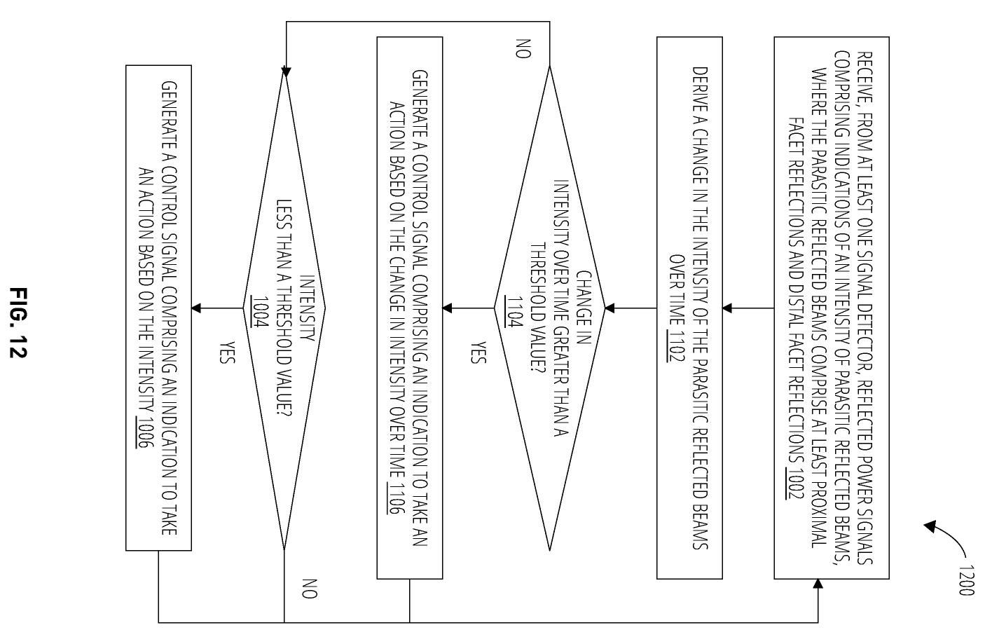

Turning now to FIG. 12 and the method 1200, which is further described with reference to method 1000 of FIG. 10 and method 1100 of FIG. 11. The method 1200 can begin at block 1002 and continue to block 1102 and then decision block 1104. From decision block 1104, method 1200 can continue to either block 1106 or decision block 1004. For example, method 1200 can continue from decision block 1104 to block 1106 where a determination is made that the change in intensity over time (or the slope of the change) is greater than the threshold value while method 1200 can continue from decision block 1104 to decision block 1004 where a determination is made that the change in intensity over time (or the slope of the change) is not greater than the threshold value. From block 1106, method 1200 can return to block 1002. Additionally, the method 1200 can continue from decision block 1004 to either block 1006 or return to block 1002. For example, method 1200 can continue from decision block 1004 to block 1006 where a determination is made that the intensity is less than the threshold value while method 1200 can continue from decision block 1004 to block 1002 where a determination is made that the intensity is not less than the threshold value.

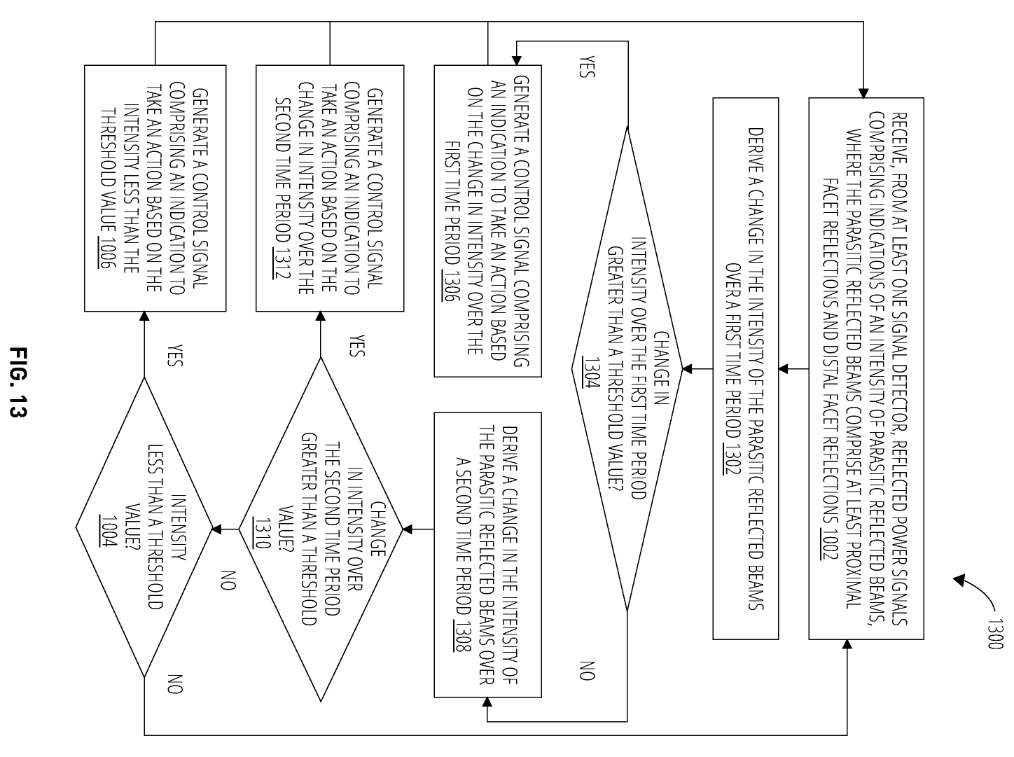

Turning now to FIG. 13 and the method 1300, which is further described with reference to method 1000 of FIG. 10. The method 1300 can begin at block 1002 and continue to block 1302. At block 1302 “derive a change in the intensity of the parasitic reflected beams over a first time period” an intensity over a first time period of the parasitic reflected beams can be determined. For example, processing unit 112 can execute instructions 128 to determine an intensity of the proximal facet reflection 908, distal facet reflection 910 and/or dark current reflections over a first time period (e.g., hundreds of seconds, minutes, or the like). As a specific example, processing unit 112 can execute instructions 128 to determine a change in of the intensity of the proximal facet reflection 908 and/or distal facet reflection 910 over a time period of 120 seconds, 240 seconds, 360 seconds, between 120 and 600 seconds. With some embodiments, processing unit 112 can execute instructions 128 to determine a change in the intensity of the proximal facet reflection 908 and/or distal facet reflection 910 over time and to further derive a slope, or rate of the change, over the time period.

Continuing to decision block 1304 “change in intensity over the first time period greater than a threshold value?” a determination can be made as to whether the change in intensity over the first time period is greater than a threshold value. For example, processing unit 112 can execute instructions 128 to determine whether the change in the intensity of the proximal facet reflection 908 and/or distal facet reflection 910 over the first time period is greater than a threshold value. Further, where at block 1302 the slope or rate of chance is derived, processing unit 112 can execute instructions 128 to determine whether the slope is greater than a threshold value. With some embodiments, the threshold values can be preset.

From decision block 1304, method 1300 can continue to block 1306 or block 1308. For example, where a determination is made that the intensity over the first time period (of the slope) is greater than the threshold value the method 1300 can continue from decision block 1304 to block 1306 while the method 1300 can continue from decision block 1304 to block 1308 where a determination is made that the intensity over the first time period (of the slope) is greater than the threshold value.

At block 1306 “generate a control signal comprising an indication to take an action based on the change in intensity over the first time period” a control signal comprising an indication to take an action based on the change in intensity over the first time period can be generated. For example, processing unit 112 can execute instructions 128 to generate a control signal comprising an indication for the surgical laser system 100 to take some action based on the change in intensity (or the slope of the change) over the first time period. In some embodiments, processing unit 112 can execute instructions 128 to generate a control signal to cause a change in operation of the surgical laser system 100 (e.g., increase pulse frequency of therapeutic laser pulses 116, increase pulse energy of therapeutic laser pulses 116, increase pulse frequency and energy of therapeutic laser pulses 116, change a suggested working distance based on measuring distance 130, or stop generation of the therapeutic laser pulses 116) to compensate for the degradation of the optical fiber 104 or to prevent damage to the surgical laser system 100. With other embodiments, processing unit 112 can execute instructions 128 to generate a control signal to cause the surgical laser system 100 to display an alert to the user (e.g., an alert suggesting a change in operating parameters such as pulse frequency and/or energy, an alert suggesting the user cleave the distal end 126 of the optical fiber 104 to renew the optical fiber 104, or an alert indicating a reduction is lasing efficiency due to degraded optical fiber 104).

For example, a large (e.g., 40%, 50%, 60%, or the like) decrease in the intensity of the proximal facet reflection 908 from the baseline can indicate severe damage to the proximal end 124 of the optical fiber 104, which can be indicative of a “dead” optical fiber that does not transmit enough laser energy. Continued lasing with such an optical fiber is not efficient and may result in damage to the surgical laser system 100 (e.g., damage to the port 228, damage to a blast shield, or the like). As another example, a small (e.g., 5%, 10%, 15%, 20%, 25%, or the like) decrease in the intensity of the proximal facet reflection 908 and/or distal facet reflection 910 from the baseline can indicate damage to the proximal end 124 and/or distal end 126 of the optical fiber 104, which may result in a corresponding decrease in the energy of the therapeutic laser pulses 116 delivered from the optical fiber 104. As such, adjusting the operational parameters of the surgical laser system 100 (e.g., corresponding increase in pulse frequency and/or energy of the therapeutic laser pulses 116) may result in a consistent delivery of energy of therapeutic laser pulses 116 ultimately delivered from the optical fiber 104. As another example, where the dark current reading increases above the baseline for a long enough time, it may indicate that the distal end 126 of the optical fiber 104 is burnt and should be cleaved to maintain transmission efficiency of the optical fiber 104.

At block 1308 “derive a change in the intensity of the parasitic reflected beams over a second time period” an intensity of the parasitic reflected beams over a second time period, different from the first time period, can be determined. For example, processing unit 112 can execute instructions 128 to determine an intensity of the proximal facet reflection 908, distal facet reflection 910 and/or dark current reflections over a second time period (e.g., hundreds of seconds, minutes, or the like) where the second time period is different from the first time period. As a specific example, processing unit 112 can execute instructions 128 to determine a change in of the intensity of the proximal facet reflection 908 and/or distal facet reflection 910 over a time period of 120 seconds, 240 seconds, 360 seconds, between 120 and 600 seconds. With some embodiments, processing unit 112 can execute instructions 128 to determine a change in the intensity of the proximal facet reflection 908 and/or distal facet reflection 910 over time and to further derive a slope, or rate of the change, over the time period.

Continuing to decision block 1310 “change in intensity over the second time period greater than a threshold value?” a determination can be made as to whether the change in intensity over the second time period is greater than a threshold value. For example, processing unit 112 can execute instructions 128 to determine whether the change in the intensity of the proximal facet reflection 908 and/or distal facet reflection 910 over the second time period is greater than a threshold value. Further, where at block 1302 the slope or rate of chance is derived, processing unit 112 can execute instructions 128 to determine whether the slope is greater than a threshold value. With some embodiments, the threshold values can be preset.

From decision block 1310, method 1300 can continue to block 1312 or decision block 1004. For example, where a determination is made that the intensity over the second time period (of the slope) is greater than the threshold value the method 1300 can continue from decision block 1310 to block 1312 while the method 1300 can continue from decision block 1310 to decision block 1004 where a determination is made that the intensity over the second time period (of the slope) is greater than the threshold value.

At block 1312 “generate a control signal comprising an indication to take an action based on the change in intensity over the second time period” a control signal comprising an indication to take an action based on the change in intensity over the second time period can be generated. For example, processing unit 112 can execute instructions 128 to generate a control signal comprising an indication for the surgical laser system 100 to take some action based on the change in intensity (or the slope of the change) over the second time period. In some embodiments, processing unit 112 can execute instructions 128 to generate a control signal to cause a change in operation of the surgical laser system 100 (e.g., increase pulse frequency of therapeutic laser pulses 116, increase pulse energy of therapeutic laser pulses 116, increase pulse frequency and energy of therapeutic laser pulses 116, change a suggested working distance based on measuring distance 130, or stop generation of the therapeutic laser pulses 116) to compensate for the degradation of the optical fiber 104 or to prevent damage to the surgical laser system 100. With other embodiments, processing unit 112 can execute instructions 128 to generate a control signal to cause the surgical laser system 100 to display an alert to the user (e.g., an alert suggesting a change in operating parameters such as pulse frequency and/or energy, an alert suggesting the user cleave the distal end 126 of the optical fiber 104 to renew the optical fiber 104, or an alert indicating a reduction is lasing efficiency due to degraded optical fiber 104).

For example, a large (e.g., 40%, 50%, 60%, or the like) decrease in the intensity of the proximal facet reflection 908 from the baseline can indicate severe damage to the proximal end 124 of the optical fiber 104, which can be indicative of a “dead” optical fiber that does not transmit enough laser energy. Continued lasing with such an optical fiber is not efficient and may result in damage to the surgical laser system 100 (e.g., damage to the port 228, damage to a blast shield, or the like). As another example, a small (e.g., 5%, 10%, 15%, 20%, 25%, or the like) decrease in the intensity of the proximal facet reflection 908 and/or distal facet reflection 910 from the baseline can indicate damage to the proximal end 124 and/or distal end 126 of the optical fiber 104, which may result in a corresponding decrease in the energy of the therapeutic laser pulses 116 delivered from the optical fiber 104. As such, adjusting the operational parameters of the surgical laser system 100 (e.g., corresponding increase in pulse frequency and/or energy of the therapeutic laser pulses 116) may result in a consistent delivery of energy of therapeutic laser pulses 116 ultimately delivered from the optical fiber 104. As another example, where the dark current reading increases above the baseline for a long enough time, it may indicate that the distal end 126 of the optical fiber 104 is burnt and should be cleaved to maintain transmission efficiency of the optical fiber 104.

From decision block 1004, the method 1300 can continue to either block 1006 or return to block 1002. For example, method 1300 can continue from decision block 1004 to block 1006 where a determination is made that the intensity is less than the threshold value while method 1300 can return to block 1002 from decision block 1004 where a determination is made that the intensity is not less than the threshold value. Further, from block 1006 and block 1312, the method 1300 can return to block 1002.

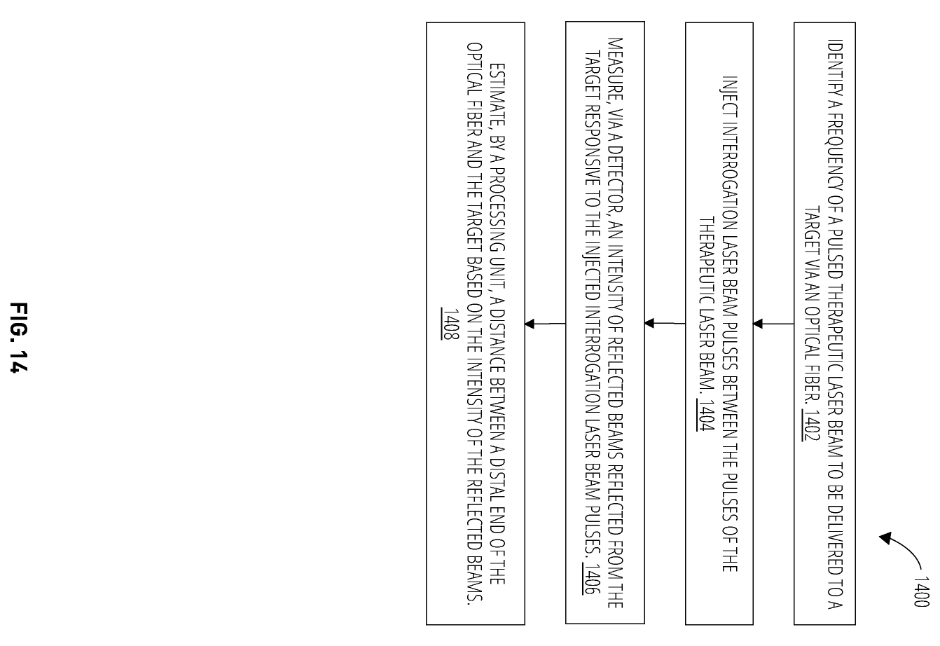

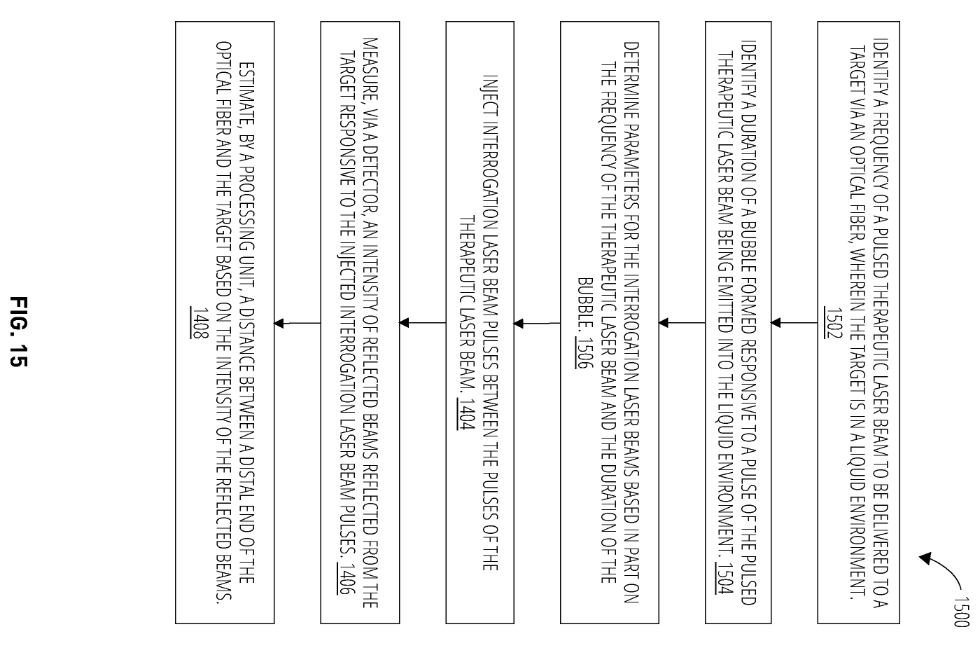

FIGS. 14 and 15 illustrate, respectively, flowcharts showing methods 1400 and 1500, for synchronizing an interrogation beam and a therapeutic beam in accordance with some embodiments of the present disclosure. The methods 1400 and 1500 are described with reference to the surgical laser system 100 of FIG. 1A and to the interrogation sub-system 200 of FIG. 2. It is to be appreciated however, that the method 1400 and/or method 1500 could be implemented by a surgical system different than the one depicted herein. In some embodiments, the methods 1400 and/or 1500 could be implemented as part of the method 300 (e.g., to interleave interrogation laser pulses 118 and therapeutic laser pulses 116 to periodically measure the distance 130 during a lithotripsy procedure). Further, the method 1500 is described with reference to the method 1400 of FIG. 14.

Turning to FIG. 14, the method 1400 can begin at block 1402. At block 1402 “identify a frequency of a pulsed therapeutic laser beam to be delivered to a target via an optical fiber” a frequency of a therapeutic laser beam can be identified. For example, processing unit 112 can execute instructions 128 to determine a frequency of the therapeutic laser beam 220, which is delivered to the target 106 via the optical fiber 104.

Continuing to block 1404 “inject interrogation laser beam pulses between the pulses of the therapeutic laser beam” interrogation laser beam pulses are injected between the pulses of the therapeutic laser beam. For example, processing unit 112 can execute instructions 128 to synchronize the interrogation laser beams 210 with the therapeutic laser beam 220. Said differently, the processing unit 112 can execute instructions 128 to inject (or synchronize in time) pulses of interrogation laser beams 210 between pulses of therapeutic laser beam 220.

Continuing to block 1406 “measure, via a detector, an intensity of reflected beams reflected from the target responsive to the injected interrogation laser beam pulses” an intensity of beams reflected from the target, responsive to incidence of the pulses of the interrogation laser beam on the target, can be measured at a light detector (or detectors). For example, signal detectors 244a and 244b can be configured to measure an intensity of the parasitic reflected beams 240 during periods where the parasitic reflected beams 240 depends upon the interrogation laser beams 210.

Continuing to block 1408 “estimate, by a processing unit, a distance between a distal end of the optical fiber and the target based on the intensity of the reflected beams” a distance between a distal end of the optical fiber and the target can be estimated based on the intensity of the reflected beams. For example, processing unit 112 can execute instructions 128 to estimate the distance 130 between the distal end 126 of the optical fiber 104 and the target 106 based on the intensity of the parasitic reflected beams 240 measured at block 1406.

Turning to FIG. 15, the method 1500 can begin at block 1502. At block 1502 “identify a frequency of a pulsed therapeutic laser beam to be delivered to a target via an optical fiber, wherein the target is in a liquid environment” a frequency of a therapeutic laser beam can be identified. For example, processing unit 112 can execute instructions 128 to determine a frequency of the therapeutic laser beam 220, which is delivered to the target 106 via the optical fiber 104. With some embodiments, the processing unit 112 can execute instructions 128 to receive an indication of the frequency from the therapeutic laser source 108. With other embodiments, the processing unit 112 can execute instructions 128 to identify the frequency from input received via a user interface (UI) when the surgical laser console 102 is configured for the current procedure. With some embodiments, processing unit 112 can execute instructions 128 to identify (e.g., at block 1502, or the like) the pulse duration and/or the decay time of the therapeutic laser beam 220 (e.g., to avoid any parasitic reflected beams 240 being influenced by the therapeutic laser beam 220 during a sampling period).

Continuing to block 1504 “identify a duration of a bubble formed responsive to a pulse of the pulsed therapeutic laser beam being emitted into the liquid environment” a duration of a bubble formed in the liquid environment when the pulsed laser beam is emitted is identified. For example, processing unit 112 can execute instructions 128 to identify a duration of a bubble formed in the liquid environment when the therapeutic laser beam 220 is emitted from the distal end 126 of the optical fiber 104. With some embodiments, the processing unit 112 can execute instructions 128 to derive the bubble duration based in part on the frequency of the therapeutic laser pulses 116 identified at block 1502 and a power of the therapeutic laser pulses 116 (e.g., received from the therapeutic laser source 108, identified from UI input, or the like).

Continuing to block 1506 “determine parameters for the interrogation laser beams based in part on the frequency of the therapeutic laser beam and the duration of the bubble” parameters for the interrogation laser beams can be determined based in part on the frequency of the therapeutic laser beam and the duration of the bubble. For example, processing unit 112 can execute instructions 128 to determine parameters for the interrogation laser beams 210 based on the identified frequency of the therapeutic laser beam 220 and the duration of the bubble formed in the liquid environment when the therapeutic laser beam 220 is emitted from the distal end 126 of the optical fiber 104.

In some embodiments, memory 114 can store optional parameters for the interrogation laser beams 210. For example, memory 114 can store an indication of a minimum and maximum frequency of the interrogation laser beams 210, a rise time of the interrogation laser beams 210, and a sampling time of the reference detector 212, signal detector 244a, signal detector 244b, and/or light snatch detector 248.

Accordingly, processing unit 112 can execute instructions 128 to determine parameters (e.g., frequency, etc.) for the interrogation laser beams 210 based on the frequency of the therapeutic laser beam 220, the decay time of the therapeutic laser beam 220, the duration of the bubble formed in the liquid environment by the therapeutic laser beam 220, the frequency range (e.g., minimum to maximum, etc.) of the interrogation laser beams 210, the rise time of the interrogation laser beams 210, and the sampling time of the detectors.

It is to be appreciated that the processing unit 112 can execute instructions 128 to determine parameters for the interrogation laser beams 210 such that (1) the interrogation laser beams 210 are transmitted through the liquid environment and not a bubble (e.g., vapor channel) formed by the therapeutic laser beam 220; (2) the therapeutic laser beam 220 does not influence the parasitic reflected beams 240; and (3) the interrogation laser beams 210 have time to reach steady state. In some embodiments, the processing unit 112 can execute instructions 128 to determine parameters for the interrogation laser beams 210 such that the interrogation laser beams 210 (e.g., laser beams 204a, 204b, and 204c, or the like) operate at the maximum possible frequency from the range of possible frequencies.

The method 1500 can continue from block 1506 to block 1404. For example, at block 1404, processing unit 112 can execute instructions 128 to send a control signal to the laser sources 202a, 202b, and 202c to cause the laser sources to generate laser beams 204a, 204b, and 204c having the parameters determined at block 1506.

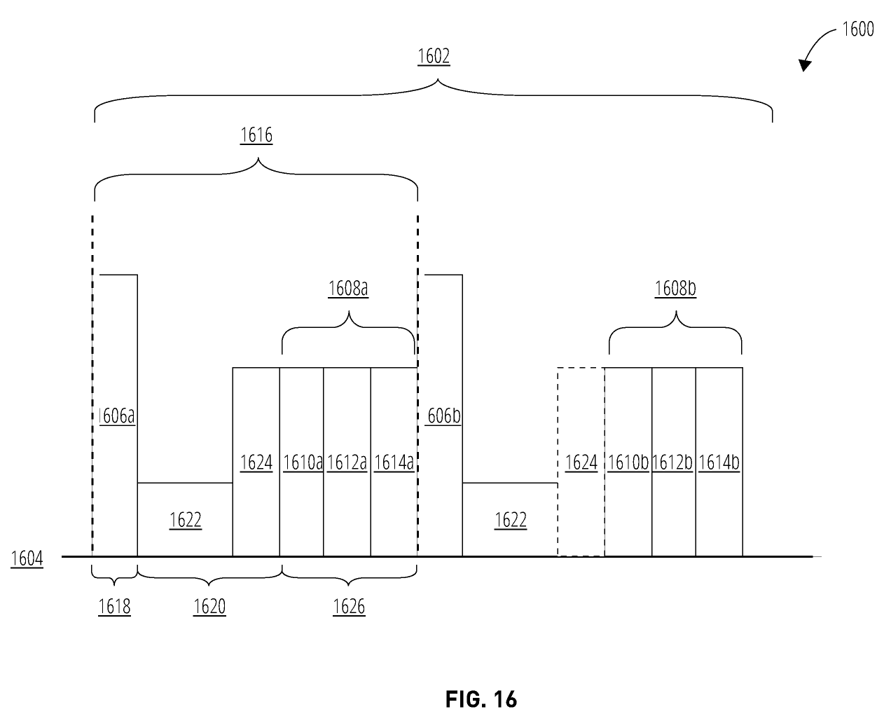

FIGS. 16, 17, 18, and 19 illustrate, respectively, charts 1600, 1700, 1800, and 1900 showing portions of example combined beams 1602, 1702, 1802, and 1902, according to embodiments of the disclosure. With some embodiments, the combined beams 1602, 1702, 1802, and 1902 can be the combined beam 224 (e.g., comprising at least interrogation laser beams 210 and therapeutic laser beam 220) as depicted in FIG. 2. It is noted that these figures are often described with reference to each other and with reference to the interrogation sub-system 200 of FIG. 2.

Turning to FIG. 16, although the combined beam 1602 can correspond to a therapeutic laser beam 220 having any of a variety of frequencies, it is contemplated that the combined beam 1602 is suitable for therapeutic laser beams 220 having frequencies between 60 and 80 Hertz (Hz). In such an example, a single pulse (or set of component pulses) of the interrogation laser beams 210 may be inserted between each pulse of the therapeutic laser beam 220.

As depicted, the combined beam 1602 is depicted against the x-axis 1604, which represents time. In general, as described above, the combined beam 1602 is composed of pulses of the therapeutic laser beam 220 and the interrogation laser beams 210. For example, therapeutic laser beam pulses 1606a and 1606b as well as interrogation beam pulses 1608a and 1608b are shown. As described above, the interrogation beam pulses 1608a and 1608b can include multiple components. For example, interrogation beam pulse 1608a is depicted including interrogation beam component A pulse 1610a, interrogation beam component B pulse 1612a, and interrogation beam component C pulse 1614a while interrogation beam pulse 1608b is depicted including interrogation beam component A pulse 1610b, interrogation beam component B pulse 1612b, and interrogation beam component C pulse 1614b.

The therapeutic laser beam pulses 1606a and 1606b may correspond to the therapeutic laser beam 220, which has a therapeutic beam frequency 1616 (e.g., between 60 and 80 Hz, or the like). Further, the therapeutic laser beam pulses therapeutic laser beam pulse 1606a and 1606b have a therapeutic pulse duration 1618. To ensure that the interrogation laser beams 210 are transmitted through the liquid environment and not a bubble (e.g., vapor channel) formed by the therapeutic laser beam 220, the therapeutic laser beam pulse 1606a and interrogation beam pulse 1608a as well as the therapeutic laser beam pulse 1606b and interrogation beam pulse 1608b are separated by a delay 1620. The delay 1620 may correspond to the bubble decay time 1622 and/or to the decay time of the therapeutic laser beam pulses 1606a and 1606b.

Further, to ensure that the therapeutic laser beam pulses therapeutic laser beam pulse 1606a and 1606b do not influence the parasitic reflected beams 240 and the interrogation beam pulses interrogation beam pulses 1608a and 1608b have time to reach steady state, the delay 1620 may include an additional time offset 1624. In some embodiments, the offset 1624 can correspond to the ramp time of the interrogation laser beams 210 and/or the decay time of the therapeutic laser beam 220.

As described above, the disclosure provides to measure the power of the interrogation laser beams 210 and the parasitic reflected beams 240 during a detection period 1626 (or sampling period). Accordingly, the detection period 1626 can correspond to the period in which signals from detectors (e.g., 212, 244a, 244b, and/or 248) are received.

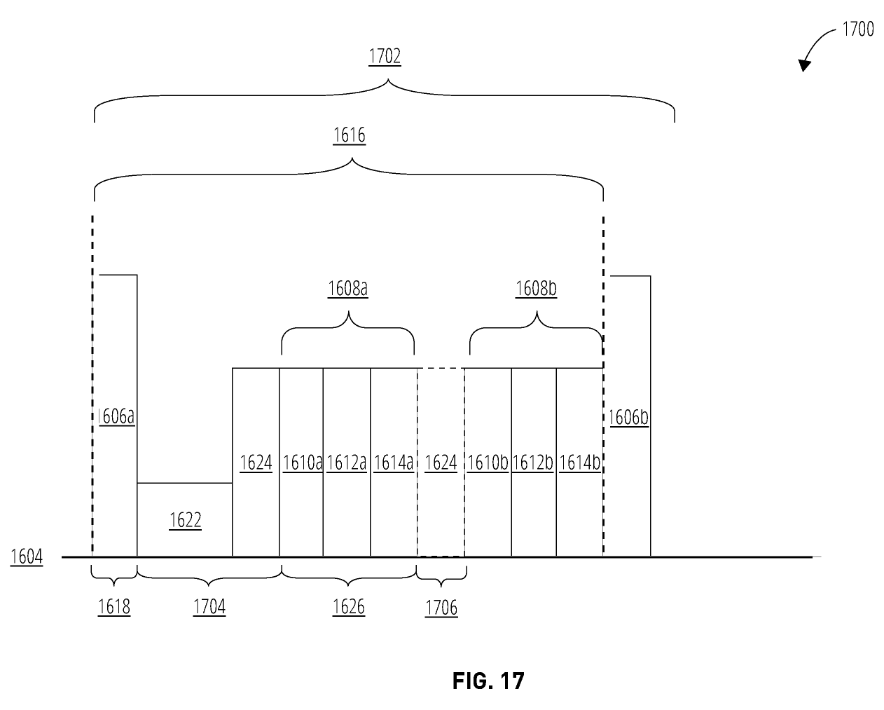

Turning to FIG. 17 and the chart 1700, which shows the combined beam 1702. Although the combined beam 1702 can correspond to a therapeutic laser beam 220 having any of a variety of frequencies, it is contemplated that the combined beam 1702 is suitable for therapeutic laser beams 220 having frequencies less than 60 Hz. In such an example, multiple pulses (or sets of component pulses) of the interrogation laser beams 210 may be inserted between each pulse of the therapeutic laser beam 220.

The combined beam 1702 is depicted against the x-axis 1604, which represents time. In general, as described above, the combined beam 1702 is composed of pulses of the therapeutic laser beam 220 and the interrogation laser beams 210. For example, therapeutic laser beam pulses 1606a and 1606b as well as interrogation beam pulses 1608a and 1608b are shown. As described above, the interrogation beam pulses 1608a and 1608b can include multiple components. For example, interrogation beam pulse 1608a is depicted including interrogation beam component A pulse 1610a, interrogation beam component B pulse 1612a, and interrogation beam component C pulse 1614a while interrogation beam pulse 1608b is depicted including interrogation beam component A pulse 1610b, interrogation beam component B pulse 1612b, and interrogation beam component C pulse 1614b.

The therapeutic laser beam pulses 1606a and 1606b may correspond to the therapeutic laser beam 220, which has a therapeutic beam frequency 1616 (e.g., less than 60 Hz, or the like). Further, the therapeutic laser beam pulses 1606a and 1606b have a therapeutic pulse duration 1618. To ensure that the interrogation laser beams 210 are transmitted through the liquid environment and not a bubble (e.g., vapor channel) formed by the therapeutic laser beam 220, the first set of pulses of the interrogation laser beams 210 following a pulse of the therapeutic laser beam 220 are initiated after an initial delay 1704 comprising the bubble decay time 1622 and the offset 1624. For example, the interrogation beam pulses 1608a are depicted separated from the therapeutic laser beam pulse 1606a by the initial delay 1704.

Optionally (e.g., as depicted) each set of pulses of the interrogation laser beams 210 can be separated by another offset 1624. In some embodiments, the dark current component described herein can be measured during the offset 1624. With some embodiments a single offset 1624 is provided at the start of each procedure and is not repeated. With some embodiments, at least one offset 1624 can be inserted between each pulse of therapeutic laser beam 220. Further, this figure depicts detection periods 1626.

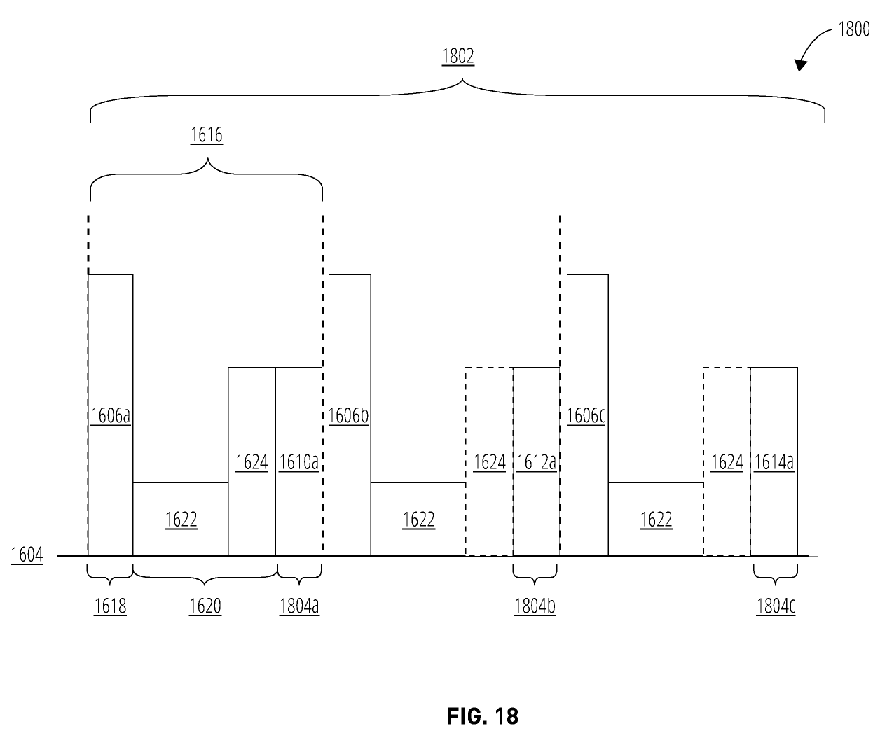

Turning to FIG. 18 and the chart 1800, which shows the combined beam 1802. Although the combined beam 1802 can correspond to a therapeutic laser beam 220 having any of a variety of frequencies, it is contemplated that the combined beam 1802 is suitable for therapeutic laser beams 220 having a frequency greater than 80 Hz or frequencies between 80 Hz and 90 Hz. In such an example, a single component pulse of the interrogation laser beams 210 may be inserted between each pulse of the therapeutic laser beam 220.

The combined beam 1802 is depicted against the x-axis 1604, which represents time. In general, as described above, the combined beam 1802 is composed of pulses of the therapeutic laser beam 220 and the interrogation laser beams 210. For example, therapeutic laser beam pulses 1606a, 1606b, and 1606c as well as interrogation beam pulses 1608a are shown. As described above, the interrogation beam pulses 1608a can include multiple components. For example, interrogation beam pulse 1608a is depicted including interrogation beam component A pulse 1610a, interrogation beam component B pulse 1612a, and interrogation beam component C pulse 1614a.

However, unlike the charts 1600 and chart 1700, each component of the interrogation beam pulse 1608a shown in chart 1800 is injected between different pulses of the therapeutic laser beam 220. For example, interrogation beam component A pulse 1610a is depicted injected between therapeutic laser beam pulses 1606a and 1606b, interrogation beam component B pulse 1612a is depicted injected between therapeutic laser beam pulses 1606b and 1606c, and interrogation beam component C pulse 1614a is depicted injected after therapeutic laser beam pulse 1606c.

Further, each component pulse of interrogation beam pulses 1608a is separated from the prior pulse of therapeutic laser beam 220 by delay 1620. Accordingly, the detection period 1626 for a single sampling or measurement of the distance between the distal end 126 and the optical fiber 104 and the target 106 may comprise three separate detection periods (e.g., detection periods 1804a, 1804b, and 1804c, or the like).

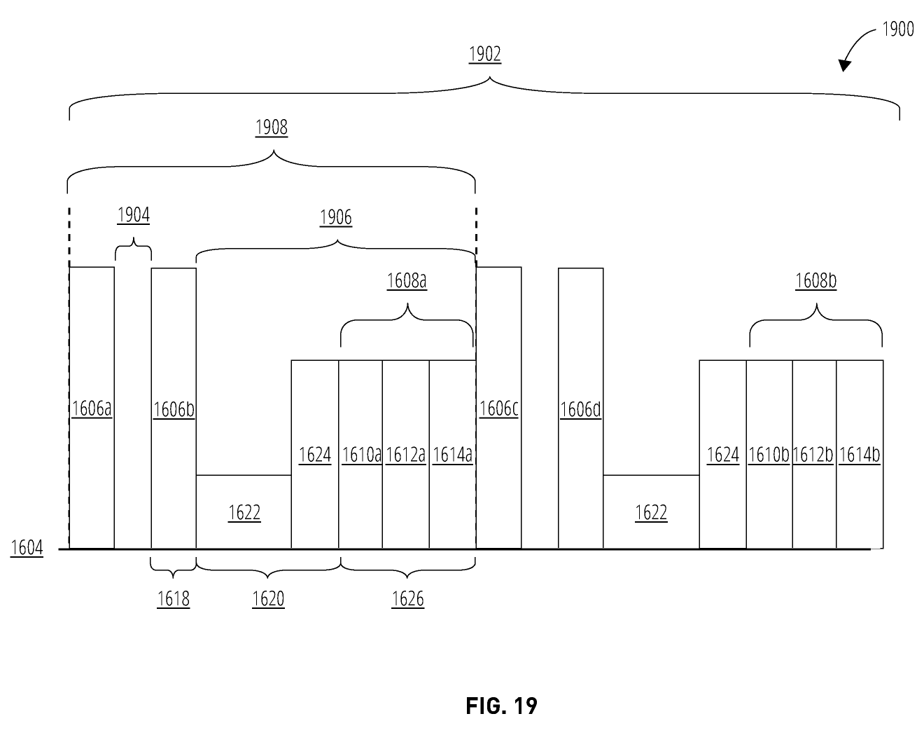

Turning to FIG. 19 and the chart 1900, which shows the combined beam 1902. Although the combined beam 1902 can correspond to a therapeutic laser beam 220 having any of a variety of frequencies, it is contemplated that the combined beam 1902 is suitable for therapeutic laser beams 220 having frequencies greater than 90 Hz, such as, for example, where multiple laser sources are used to generate a therapeutic laser beam 220 having an enhanced frequency rate (EFR).

In such an example, a group (e.g., two, three, four, etc.) pulses of the therapeutic laser beam 220 can be separated by a short delay while each group is separated by a longer delay. For example, a first group of laser pulses comprising therapeutic laser beam pulses 1606a and 1606b and a second group of laser pulses comprising therapeutic laser beam pulses 1606c and 1606d are shown. The pulses from the first group (e.g., therapeutic laser beam pulse 1606a and 1606b) are separated by intra-group delay 1904 while adjacent pulses from each group (e.g., therapeutic laser beam pulse 1606b and 1606c) are separated by group delay 1906. In such a manner, the frequency 1908 corresponds to the time between groups multiplied by the number of pulses in each group (e.g., two as is the case depicted in FIG. 19).

As can be seen, the intra-group delay 1904 is shorter than the group delay 1906. As such, the component pulses of the interrogation laser beams 210 can be injected between adjacent pulses from each group. For example, interrogation beam pulses 1608a are injected between therapeutic laser beam pulses 1606b and 1606c while interrogation beam pulses 1608b are injected between therapeutic laser beam pulses 1606d and the next pulse of therapeutic laser beam 220 (not shown). Further, as depicted, the interrogation beam pulses 1608a and 1608b are separated from the prior pulse of the therapeutic laser beam 220 by delay 1620.

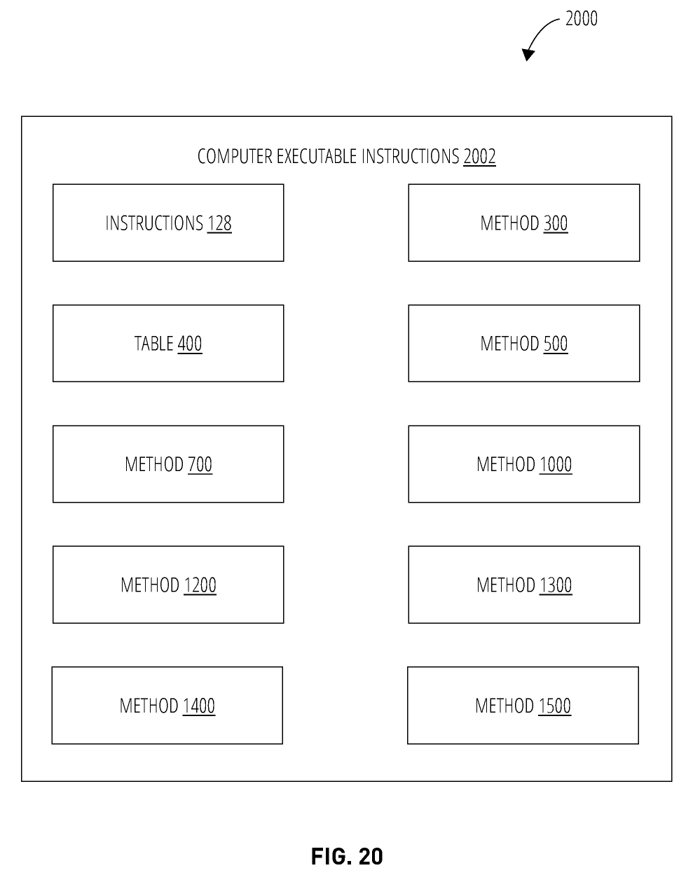

FIG. 20 illustrates computer-readable storage medium 2000. Computer-readable storage medium 2000 may comprise any non-transitory computer-readable storage medium or machine-readable storage medium, such as an optical, magnetic or semiconductor storage medium. In various embodiments, computer-readable storage medium 2000 may comprise an article of manufacture. In some embodiments, computer-readable storage medium 2000 may store computer executable instructions 2002 with which circuitry (e.g., processing unit 112, or the like) of a surgical laser system or console (e.g., surgical laser console 102) can execute. For example, computer executable instructions 2002 can include instructions to implement operations described with respect to instructions 128, method 300, table 400, method 500, method 700, method 1000, method 1100, method 1200, method 1300, method 1400, and/or method 1500. Examples of computer-readable storage medium 2000 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 2002 may include any suitable type of code, such as source code, compiled code, interpreted code, executable code, static code, dynamic code, object-oriented code, visual code, and the like.

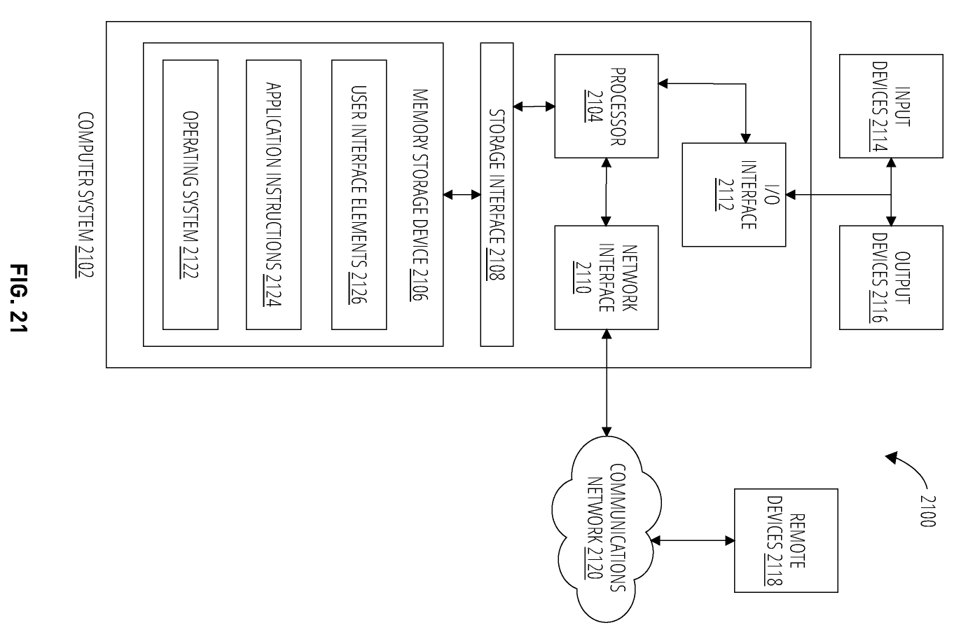

FIG. 21 is a block diagram of a computing environment 2100 including a computer system 2102 for implementing embodiments consistent with the present disclosure. In some embodiments, the computing environment 2100, or portion thereof (e.g., the computer system 2102) may comprise or be comprised in a surgical laser console (e.g., surgical laser console 102, or the like). Accordingly, in various embodiments, computer system 2102 may be used to derive a distance 130 between the distal end 126 of the optical fiber 104 and the target 106 while accounting for parasitic reflections as outlined above.

The computer system 2102 may include a central processing unit (“CPU” or “processor”) 2104. The processor 2104 may include at least one data processor for executing instructions and/or program components for executing user or system-generated processes. A user may include a person, a person using a device such as those included in this disclosure, or another device. The processor 2104 may include specialized processing units such as integrated system (bus) controllers, memory management control units, floating point units, graphics processing units, neural processing units, digital signal processing units, etc. The processor 2104 may be disposed in communication with input devices 2114 and output devices 2116 via I/O interface 2112. The I/O interface 2112 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n /b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMax, or the like), etc.

Using the I/O interface 2112, computer system 2102 may communicate with input devices 2114 and output devices 2116. In some embodiments, the processor 2104 may be disposed in communication with a communications network 2120 via a network interface 2110. In various embodiments, the communications network 2120 may be utilized to communicate with a remote memory storage device 2106, such as for accessing look-up tables, performing updates, or utilizing external resources. The network interface 2110 may communicate with the communications network 2120. The network interface 2110 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 2120 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 2120 may include a variety of network devices, including routers, bridges, servers, computing devices, storage devices, etcetera. In some embodiments, the processor 2104 may be disposed in communication with a memory storage device 2106 via a storage interface 2108. The storage interface 2108 may connect to memory storage device 2106 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 2106 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 2106 may store a collection of program or database components, including, without limitation, an operating system 2122, application instructions 2124, and user interface elements 2126. In various embodiments, the operating system 2122 may facilitate resource management and operation of the computer system 2102. Examples of operating systems include, without limitation, APPLE^{®} MACINTOSH^{®} OS X^{®}, UNIX^{®}, UNIX-like system distributions (E.G., BERKELEY SOFTWARE DISTRIBUTION^{®} (BSD), FreeBSD^{®}, NetBSD^{®}, OpenBSD^{®}, etc.), LINUX^{®} DISTRIBUTIONS (E.G., RED HAT^{®}, UBUNTU^{®}, KUBUNTU^{®}, etc.), IBM^{®}OS/2^{®}, MICROSOFT^{®} WINDOWS^{®} (XP^{®}, VISTA^{®}/7/8, 10 etc.), APPLE^{®} IOS^{®}, GOOGLE^{TM} ANDROID^{TM}, BLACKBERRY^{®} OS, or the like.

The application instructions 2124 may include instructions that when executed by the processor 2104 cause the processor 2104 to perform one or more techniques, steps, procedures, and/or methods described herein, such to irrigate a site and irradiate a site as outlined herein. For example, application instructions 2124, when executed by processor 2104 can cause processor 2104 to perform the method 300.

The user interface elements 2126 may facilitate display, execution, interaction, manipulation, or operation of program components through textual or graphical facilities. For example, user interfaces may provide computer interaction interface elements on a display system operatively connected to the computer system 2102, such as cursors, icons, checkboxes, menus, scrollers, windows, widgets, etcetera. The user interface elements 2126 may be employed by application instructions 2124 and/or operating system 2122 to provide, for example, a user interface with which a user can interact with computer system 2102. As a specific example, the distance 130 derived as outlined herein can be displayed on a display. In some embodiments, the user interface elements 2126 may be integrated with the display (not shown).

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to a single one or multiple ones. Additionally, the words "herein," "above," "below" and words of similar import, when used in this application, refer to this application as a whole and not to any particular portions of this application. When the claims use the word "or" in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

What is claimed is:

1. A surgical laser system comprising:
at least one laser source configured to output an interrogation laser beam, the interrogation laser beam comprising a plurality of wavelength components;
a reference detector configured to measure an intensity of the interrogation laser beam at each of the plurality of wavelength components;
at least one signal detector configured to measure an intensity of a reflected light beam at each of the plurality of wavelength components;
a processor; and
a memory comprising instructions, which when executed by the processor cause the surgical laser system to:
generate, via one or more of the at least one laser source, an interrogation laser beam, the interrogation laser beam comprising a plurality of wavelength components;
measure, via the reference detector, an intensity of the interrogation laser beam at each of the wavelength components;
illuminate, via an optical fiber coupled to the surgical laser system, a target with the interrogation laser beam;
measure, via the at least one signal detector, an intensity of a reflected light beam at each of the plurality of wavelength components, wherein the reflected light beam comprises target reflections and parasitic reflections;
derive for each of the measured intensities, a normalized intensity based on the measured intensity and a dark current intensity associated with the measured intensity;
derive for each of the measured intensities of the reflected light beam, a corrected intensity based on the normalized intensity and one or more parasitic intensities; and
estimate a distance between a distal end of the optical fiber and the target based on the corrected intensities.

2. The surgical laser system of claim 1, the instructions when executed by the processor further cause the surgical laser system to apply a smoothing filter to each of the measured intensities prior to deriving the normalized intensities.

3. The surgical laser system of claim 1, wherein the first wavelength component has a lower water absorption coefficient than the second wavelength component.

4. The surgical laser system of claim 1, wherein the plurality of wavelength components comprises a first wavelength component, a second wavelength component, and a third wavelength component.

5. The surgical laser system of claim 4, wherein the first wavelength component is 1310 nanometers (nm), wherein the second wavelength component is 1340 nm, and wherein the third wavelength component is 1431 nm.

6. The surgical laser system of claim 4, the instructions when executed by the processor further cause the surgical laser system to:
derive a first non-calibration corrected signal for the measured intensity of the reflected light beam at the first wavelength component based on the normalized intensity and a first distal facet reflection value, the first distal facet reflection value associated with the first wavelength component;
derive a second non-calibration corrected signal for the measured intensity of the reflected light beam at the second wavelength component based on the normalized intensity and a second distal facet reflection value, the second distal facet reflection value associated with the second wavelength component;
derive a first calibration corrected signal for the measured intensity of the reflected light beam at the first wavelength component based on the normalized intensity, the first distal facet reflection value, the normalized intensity of the reflected light beam at the third wavelength component, and a third distal facet reflection value, the third distal facet reflection value associated with the third wavelength component; and
derive a second calibration corrected signal for the measured intensity of the reflected light beam at the second wavelength component based on the normalized intensity, the second distal facet reflection value, the normalized intensity of the reflected light beam at the third wavelength component, and the third distal facet reflection value.

7. The surgical laser system of claim 6, the instructions when executed by the processor further cause the surgical laser system to derive, for each of the first and the second non-calibration corrected signals, corrected signals based on a difference between the internal reflections and the last known fiber distal end reflection normalized signals, respectively.

8. The surgical laser system of claim 7, the instructions when executed by the processor further cause the surgical laser system to:
derive a non-calibration corrected distance based on the first and second non-calibration corrected signals; and
derive a calibration corrected distance based on the first and second calibration corrected signals.

9. The surgical laser system of claim 8, the instructions when executed by the processor further cause the surgical laser system to derive the non-calibration corrected distance and the calibration corrected distance are derived based on fundamental physical equations incorporating the difference between water absorption coefficients of the first wavelength component and the second wavelength component.

10. The surgical laser system of claim 9, the instructions when executed by the processor further cause the surgical laser system to:
determine that the estimated distance is zero (o);
determine that the estimated distance is infinity;
select the non-calibration corrected distance as the estimated distance; or
select the calibration corrected distance as the estimated distance.

11. The surgical laser system of claim 10, the instructions when executed by the processor further cause the surgical laser system to:
determine whether the non-calibration corrected distance is greater than zero;
determine whether the calibration corrected distance is greater than zero; and
determine that the estimated distance is zero based on a determination that the non-calibration corrected distance is not greater than zero and a determination that the calibration corrected distance is not greater than zero.

12. The surgical laser system of claim 11, the instructions when executed by the processor further cause the surgical laser system to:
determine whether the non-calibration corrected distance is less than a maximum measurable distance;
determine whether the calibration corrected distance is less than a maximum measurable distance; and
determine that the estimated distance is infinity based on a determination that the non-calibration corrected distance is not less than the maximum measurable distance and a determination that the calibration corrected distance is not less than the maximum measurable distance; or
determine that the estimated distance is infinity based on a determination that the calibration corrected distance is less than zero and a determination that the non-calibration corrected distance is not less than the maximum measurable distance.

13. The surgical laser system of claim 12, the instructions when executed by the processor further cause the surgical laser system to select the non-calibration corrected distance as the estimated distance based on a determination that the calibration corrected distance is not greater than zero, a determination that the non-calibration corrected distance is greater than zero, and a determination that the non-calibration corrected distance is less than the maximum measurable distance.

14. The surgical laser system of claim 12, the instructions when executed by the processor further cause the surgical laser system to:
derive a calibration ratio based on the normalized intensity of the reflected light beam at the third wavelength component and the third distal facet reflection value;
determine whether the calibration ratio is greater than a calibration ratio threshold; and
selecting the non-calibration corrected distance as the estimated distance based on:
a determination that the calibration ratio is greater than the calibration ratio threshold, and
a determination that the calibration corrected distance is greater than zero and a determination that the calibration corrected distance is not greater than the maximum measurable distance, or
a determination that the calibration corrected distance is greater than the maximum measurable distance and a determination that the non-calibration corrected distance is not greater than the maximum measurable distance; or
select the calibration corrected distance as the estimated distance based on:
a determination that the calibration ratio is not greater than the calibration ratio threshold, and
a determination that the calibration corrected distance is greater than zero and a determination that the calibration corrected distance is not greater than the maximum measurable distance, or
a determination that the calibration corrected distance is greater than the maximum measurable distance and a determination that the non-calibration corrected distance is not greater than the maximum measurable distance.

15. At least one non-transitory machine-readable storage device comprising instructions, which when executed by a processor of a surgical laser system cause the surgical laser system to:
generate, via one or more of laser sources of the surgical laser system, an interrogation laser beam, the interrogation laser beam comprising a plurality of wavelength components;
measure, via a reference detector of the surgical laser system, an intensity of the interrogation laser beam at each of the wavelength components;
illuminate, via an optical fiber coupled to the surgical laser system, a target with the interrogation laser beam;
measure, via at least one signal detector of the surgical laser system, an intensity of a reflected light beam at each of the plurality of wavelength components, wherein the reflected light beam comprises target reflections and parasitic reflections;
derive for each of the measured intensities, a normalized intensity based on the measured intensity and a dark current intensity associated with the measured intensity;
derive for each of the measured intensities of the reflected light beam, a corrected intensity based on the normalized intensity and one or more parasitic intensities; and
estimate a distance between a distal end of the optical fiber and the target based on the corrected intensities.

16. The at least one non-transitory machine-readable storage device of claim 15, wherein the plurality of wavelength components comprises a first wavelength component, a second wavelength component, and a third wavelength component and wherein the instructions when executed by the processor further cause the surgical laser system to:
derive a first non-calibration corrected signal for the measured intensity of the reflected light beam at the first wavelength component based on the normalized intensity and a first distal facet reflection value, the first distal facet reflection value associated with the first wavelength component;
derive a second non-calibration corrected signal for the measured intensity of the reflected light beam at the second wavelength component based on the normalized intensity and a second distal facet reflection value, the second distal facet reflection value associated with the second wavelength component;
derive a first calibration corrected signal for the measured intensity of the reflected light beam at the first wavelength component based on the normalized intensity, the first distal facet reflection value, the normalized intensity of the reflected light beam at the third wavelength component, and a third distal facet reflection value, the third distal facet reflection value associated with the third wavelength component; and
derive a second calibration corrected signal for the measured intensity of the reflected light beam at the second wavelength component based on the normalized intensity, the second distal facet reflection value, the normalized intensity of the reflected light beam at the third wavelength component, and the third distal facet reflection value.

17. The at least one non-transitory machine-readable storage device of claim 16, the instructions when executed by the processor further cause the surgical laser system to derive, for each of the first and the second non-calibration corrected signals, corrected signals based on a difference between the internal reflections and the last known fiber distal end reflection normalized signals, respectively.

18. The at least one non-transitory machine-readable storage device of claim 17, the instructions when executed by the processor further cause the surgical laser system to derive the non-calibration corrected distance and the calibration corrected distance are derived based on fundamental physical equations incorporating the difference between water absorption coefficients of the first wavelength component and the second wavelength component.

19. A method for a surgical laser system, comprising:
generating, via a laser source, an interrogation laser beam, the interrogation laser beam comprising a plurality of wavelength components;
measuring, via a reference detector, an intensity of the interrogation laser beam at each of the wavelength components;
illuminating, via an optical fiber coupled to the surgical laser system, a target with the interrogation laser beam;
measuring, via at least one signal detectors, an intensity of a reflected light beam at each of the plurality of wavelength components, wherein the reflected light beam comprises target reflections and parasitic reflections;
deriving for each of the measured intensities, a normalized intensity based on the measured intensity and a dark current intensity associated with the measured intensity;
deriving for each of the measured intensities of the reflected light beam, a corrected intensity based on the normalized intensity and one or more parasitic intensities; and
estimating a distance between a distal end of the optical fiber and the target based on the corrected intensities.

20. The method of claim 19, wherein the plurality of wavelength components comprises a first wavelength component, a second wavelength component, and a third wavelength component, and
wherein deriving for each of the measured intensities of the reflected light beam, a corrected intensity comprises:
deriving a first non-calibration corrected signal for the measured intensity of the reflected light beam at the first wavelength component based on the normalized intensity and a first distal facet reflection value, the first distal facet reflection value associated with the first wavelength component;
deriving a second non-calibration corrected signal for the measured intensity of the reflected light beam at the second wavelength component based on the normalized intensity and a second distal facet reflection value, the second distal facet reflection value associated with the second wavelength component;
deriving a first calibration corrected signal for the measured intensity of the reflected light beam at the first wavelength component based on the normalized intensity, the first distal facet reflection value, the normalized intensity of the reflected light beam at the third wavelength component, and a third distal facet reflection value, the third distal facet reflection value associated with the third wavelength component; and
deriving a second calibration corrected signal for the measured intensity of the reflected light beam at the second wavelength component based on the normalized intensity, the second distal facet reflection value, the normalized intensity of the reflected light beam at the third wavelength component, and the third distal facet reflection value.
